(19) United States
(12) Patent Application Publication
(10) Pub. No.: US 2024/0010655 A1
(43) Pub. Date: Jan. 11, 2024

(54) DIHYDROIMIDAZO PYRIMIDO PYRIMIDINONE COMPOUND

(71) Applicant: IMPACT THERAPEUTICS (SHANGHAI), INC, Shanghai (CN)

(72) Inventors: Sui Xiong CAI, Shanghai (CN); Ye Edward TIAN, Shanghai (CN); Xiaozhu WANG, Nanjing (CN)

(21) Appl. No.: 17/769,416

(22) PCT Filed: Oct. 13, 2020
(86) PCT No.: PCT/CN2020/120569

§ 371 Date: Apr. 15, 2022

(30) Foreign Application Priority Data
Oct. 16, 2019 (CN) 201910987593.8

(51) Int. Cl. C07D 487/14; A61K 45/06; A61P 35/00

(52) U.S. Cl. CPC C07D 487/14; A61K 45/06; A61P 35/00

(57) ABSTRACT

Disclosed are dihydroimidazopyrimidopyrimidinone compounds, specifically represented by Formula I:

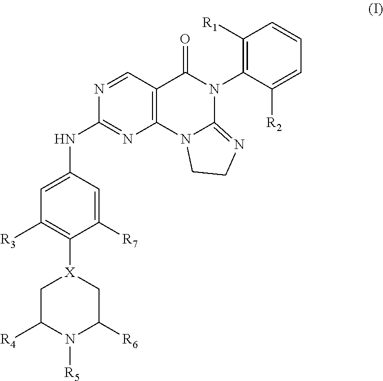

or pharmaceutically acceptable salts or prodrugs thereof, wherein the substituents are defined herein. Compounds of Formula I are Wee1 kinase inhibitors. Therefore, compounds of the invention may be used to treat diseases caused by abnormal Wee1 activity.

### FIELD OF THE DISCLOSURE

This disclosure is in the field of medicinal chemistry. In particular, the disclosure relates to 8,9-dihydroimidazo[1,2-a]pyrimido[5,4-e]pyrimidin-5(6H)-ones, and the use of these compounds as Wee1 kinase inhibitors and anti-cancer drugs.

### BACKGROUND OF THE INVENTION

The process of growth and proliferation of eukaryotic cell includes that the parent cell produces two identical daughter cells through the mitosis of the cell chromosome by accurately replicating its genome containing genetic information. This process of cell proliferation and division is called the cell cycle, and it involves the process of a cell going from one division to the next. The cell cycle consists of four growth stages: the G1 phase of massive synthesis of proteins and RNA after mitosis, the S phase of DNA synthesis and replication, the G2 phase of preparation before mitosis, and the M phase of mitosis. Cells divide and proliferate through the cell cycle, or stop, depending on the state and needs of the cell. It is necessary to keep genetic information complete and correct during cell proliferation and division. Whether or not to enter the next phase of cell cycle until the completion of the whole cell cycle is ensured and completed through the checkpoints in the cell cycle process.

During the whole process of cell cycle, there are many cell cycle checkpoints. Each cell cycle checkpoint consists of a very complex system and is composed of multiple factors. In the G1 phase, the checkpoint determines whether to enter the cell cycle by examining the state inside and outside the cell, so as to determine whether the cell enters the S phase of DNA synthesis. The G1 checkpoint is a complex system that includes the famous CDK4/CDK6. Another important checkpoint is the so-called G2-M checkpoint, where the cell completes DNA replication (S phase) and enters the cell growth phase (G2 phase). This checkpoint examines whether there is any DNA damage or defect after the cells have synthesized DNA, which determines whether the cells undergo mitosis (M-phase) with the separation of the following chromosomes. Cell cycle checkpoints at this stage include complex kinase Cdk1 complexes including Cyclin-B-cdc2 (Nurse, P., 1990, Nature 344, 503-508). Activation of Cdk1 leads to initiation of mitosis, and subsequent inactivation is accompanied by the completion of mitosis. The activity of Cdk1 is regulated by cdc2 binding to Cyclin-A or Cyclin-B and its phosphorylation. For example, the activation of the cyclin B-Cdk1 complex causes mitosis (Lindqvist, A., et al, 2009, The Journal of cell biology 185, 193-202). Cdc2 is kept inactive by phosphorylation before mitosis. Its phosphorylation state is achieved by tyrosine kinase Wee1, etc. In addition, there are M-phase cell cycle checkpoints.

Tyrosine 15 (Y15) on Cdk1 is phosphorylated by Wee1, thus inhibiting the activity of Cdk1 (McGowan, C. H., et al, 1993, The EMBO journal 12, 75-85; Parker, L. L., et al, 1992, Science 257, 1955-1957). Therefore, Wee1 is a key inhibitory regulator of Cdk1 activity and plays an important role in G2-M phase checkpoints to ensure the entry into mitosis without DNA damage after DNA replication (O'Connell, et al, 1997, The EMBO journal 16, 545-554). Loss or inactivation of Wee1 may result in premature entry into mitosis, leading to mitotic failure and cell death (Stumpff, J., et al, 2004, Curr Biol 14, 2143-2148). Some tumor cells have functional deficiency in G1 cell cycle checkpoint and rely on G2-M cell cycle checkpoints to ensure the progress of cell cycle (Sancar, A., et al, 2004, Annual review of biochemistry 73, 39-85). Due to the loss of p53 protein function, in these cancer cells, the loss of Wee1 expression or the inhibition of Wee1 activity will result in the loss of G2-M phase checkpoints, making tumor cells very sensitive to DNA damage, and this sensitivity is especially prominent in tumor cells that lose the ability of G1 phase checkpoint (Wang, Y., et al, 2004, Cancer biology & therapy 3, 305-313).

In summary, inhibition of Wee1 activity can selectively promote the death of cancer cells with defective cell cycle checkpoints; at the same time, has little effect on normal cells with normal cell cycle checkpoints. Therefore, Wee1 inhibitors may be used as targeted drugs for the treatment of cancer and other cell proliferation disorders.

In addition, because the inhibition of Wee1 activity increases the sensitivity of cells to DNA damage, Wee1 inhibitors can be used in combination with anticancer drugs that cause DNA damage or inhibit DNA repair mechanism, including PARP inhibitors, e.g. Olaparib, Niraparib, Rucaparib and Talazoparib; HDAC inhibitors, e.g. vorinotat, lomidacin, pabista, and belistatin; and the like, for treating cancer or other cell proliferation disorders. Wee1 inhibitors may also be used in combination with other anticancer drugs related to cell cycle checkpoints of cell division, including Chk1/2 inhibitors, CDK4/6 inhibitors such as Paboxini, ATM/ATR inhibitors etc. for the treatment of cancer and other diseases.

The study of Karnak et al. (Clin Cancer Res, 2014, 20(9): 5085-5096) shows that the combination of Wee1 inhibitor AZD1775 and PARP inhibitor olaparib can enhance the sensitivity of pancreatic cancer after radiotherapy. The results confirmed that the combination of Wee1 inhibitor and PARP inhibitor could enhance the radiosensitivity of pancreatic cancer, and supported the hypothesis that Wee1 inhibition could sensitize the cell to PARP inhibitor, i.e., sensitize the cell to radiotherapy by inhibiting the function of DNA repair and G2 checkpoint. It can eventually lead to the accumulation of unrepaired damaged DNA until the cell dies.

In addition, it was reported (BMC Cancer, 2015, 15: 462) that Wee1 inhibitor MK1775 and Chk1/2 inhibitor AZD7762 were used together in malignant melanoma cell and xenograft models. The results showed that the combined use of Wee1 and Chk1/2 inhibitors could synergize the inhibitory effect of single drug, thus reducing the proliferation capacity of tumor cells and activating the apoptosis mechanism. The combination of both inhibitors can inhibit tumor growth better in the xenograft model.

AZD1775 is the first Wee1 kinase inhibitor with single antitumor activity in a preclinical model. Phase I clinical studies showed the single drug efficacy of AZD1775 in patients with solid tumors with BRCA mutations, and the inhibition mechanism of Wee1 kinase was confirmed by paired tumor biopsy finding changes related to targeting and DNA damage response (J Clin Oncol, 2015, 33: 3409-3415). In a clinical phase I trial of AZD1775, which enrolled in more than 200 patients, the efficacy of AZD1775 alone or in combination with gemcitabine, cisplatin or carboplatin in the treatment of patients with advanced solid tumors was studied, showing that AZD1775 alone or in combination with chemotherapy was safe and tolerable at a certain dose. Of 176 evaluable patients, 94 (53%) had stable disease as the best response, and 17 (10%) had partial response. Importantly, the response rate of AZD1775 in patients with TP53 mutation (n=19) was 21%, while that in TP53 wild-type patients (n=33) was 12%, showing great potential for patients with TP53 mutation (J Clin Oncol, 2016 Sep. 6, pii: JCO675991).

Various kinase inhibitors have been disclosed. For example, WO2012161812 disclosed the following tricyclic compounds as Wee1 kinase inhibitors; WO2005021551 disclosed the following tetracyclic pyrimidine or pyridine compounds as protein kinase inhibitors; WO2018090939 disclosed the following dihydroimidazopyrimidopyrimidinones as Wee1 kinase inhibitors.

### SUMMARY OF THE DISCLOSURE

The disclosure provides novel 8,9-dihydroimidazo[1,2-a]pyrimido[5,4-e]pyrimidin-5(6H)-ones, as represented in Formula I (including Formulae Ia, Ib and Ic) as kinase inhibitors, especially Wee1 kinase inhibitors.

The present disclosure also provides pharmaceutical compositions comprising a compound of Formula I (including Formulae Ia, Ib and Ic) in an effective amount for the treatment of cancer.

In a particular embodiment, the pharmaceutical composition useful for the treatment of cancer may also contain one or more pharmaceutically acceptable carriers or diluents.

In a particular embodiment, the pharmaceutical composition useful for the treatment of cancer may also contain at least one known anticancer drugs or its pharmaceutically acceptable salts.

The disclosure is also directed to methods for the preparation of novel compounds of Formula I (including Formulae Ia, Ib and Ic).

### DETAILED DESCRIPTION OF THE DISCLOSURE

The disclosure finds novel 8,9-dihydroimidazo[1,2-a]pyrimido[5,4-e]pyrimidin-5(6H)-ones as kinase inhibitors, especially Wee1 kinase inhibitors, as represented in Formula I (including Formulae Ia, Ib and Ic).

Specifically, compounds of the present disclosure are represented by Formula I:

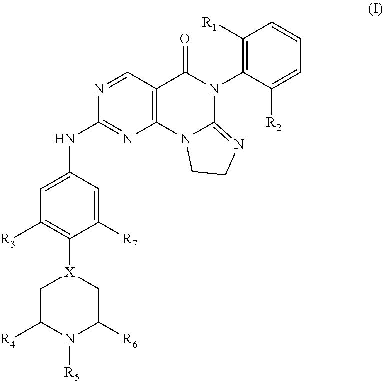

or stereoisomers, or pharmaceutically acceptable salts or prodrugs thereof, wherein:
R₁ and R₂ are independently halo; R₃ is halo, C₁₋₄ alkyl or C₁₋₄ alkoxy; R₄ and R₆ are independently H or C₁₋₄ alkyl; R₅ is H or C₁₋₄ alkyl; R₇ is H, halo, C₁₋₄ alkyl or C₁₋₄ alkoxy; and X is CH or N;
wherein, compounds of Formula I do not include the following compounds:
6-(2-chloro-6-fluorophenyl)-2-((3-fluoro-4-((3R,5S)-3,4,5-trimethylpiperazin-1-yl)phenyl) amino)-8,9-dihydroimidazo[1,2-a]pyrimido[5,4-e]pyrimidin-5(6H)-one;
6-(2-chloro-6-fluorophenyl)-2-((3-chloro-4-((3R,5S)-3,4,5-trimethylpiperazin-1-yl)phenyl)-8,9-dihydroimidazo[1,2-a]pyrimido[5,4-e]pyrimidin-5(6H)-one;
6-(2-chloro-6-fluorophenyl)-2-((3-methyl-4-((3R,5S)-3,4,5-trimethylpiperazin-1-yl)phenyl) amino)-8,9-dihydroimidazo[1,2-a]pyrimido[5,4-e]pyrimidin-5(6H)-one;
6-(2-chloro-6-fluorophenyl)-2-((4-((3S,5R)-4-isopropyl-3,5-dimethylpiperazin-1-yl)-3-methylphenyl)amino)-8,9-dihydroimidazo[1,2-a]pyrimido[5,4-e]pyrimidin-5(6H)-one;
6-(2,6-dichlorophenyl)-2-((3-fluoro-4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-chloro-4-((3S,5R)-3,4,5-trimethylpiperazin-1-yl)phenyl) amino)-8,9-dihydroimidazo[1,2-a]pyrimido[5,4-e]pyrimidin-5(6H)-one;
6-(2,6-dichlorophenyl)-2-((3-methyl-4-((3S,5R)-3,4,5-trimethylpiperazin-1-yl)phenyl) amino)-8,9-dihydroimidazo[1,2-a]pyrimido[5,4-e]pyrimidin-5(6H)-one;
6-(2,6-dichlorophenyl)-2-((3-methyl-4-((3S,5R)-4-isopropyl-3,5-dimethylpiperazin-1-yl) phenyl) amino)-8,9-dihydroimidazo[1,2-a]pyrimido[5,4-e]pyrimidin-5(6H)-one;
6-(2,6-dichlorophenyl)-2-((3,5-dichloro-4-((3S,5R)-3,4,5-trimethylpiperazin-1-yl)phenyl) amino)-8,9-dihydroimidazo[1,2-a]pyrimido[5,4-e]pyrimidin-5(6H)-one; and
6-(2,6-dichlorophenyl)-2-((3-chloro-5-methyl-4-((3S,5R)-3,4,5-trimethylpiperazin-1-yl)phenyl) amino)-8,9-dihydroimidazo[1,2-a]pyrimido[5,4-e]pyrimidin-5(6H)-one.

In the preferred embodiments of Formula I, both of R₁ and R₂ are chloro.

In the preferred embodiments of Formula I, R₃ is halo, methyl or ethyl.

In the preferred embodiments of Formula I, R₇ is H, halo, methyl or methoxy.

In the preferred embodiments of Formula I, R₄ and R₆ each are independently H or methyl.

In the preferred embodiments of Formula I, R₅ is H, methyl or methyl-d3.

In the preferred embodiments of Formula I, when X is N, R₄, R₅ and R₆ are not all H; preferably, R₄ and R₆ is C₁₋₄ alkyl, R₅ is H or C₁₋₄ alkyl; more preferably, R₄ and R₆ is methyl, R₅ is H, methyl or methyl-d3.

In the preferred embodiments of Formula I, compounds of Formula I have the structure as represented in the following Formula Ia:

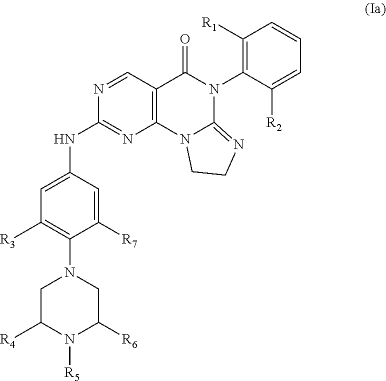

or stereoisomers, or pharmaceutically acceptable salts or prodrugs thereof, wherein:
R₁ and R₂ are independently halo; R₃ is halo or C₁₋₄ alkyl; R₇ is H, halo, C₁₋₄ alkyl or C₁₋₄ alkoxy; R₄ and R₆ each are independently C₁₋₄ alkyl; R₅ is H or C₁₋₄ alkyl; wherein, compounds of Formula Ta do not include the following compounds:
6-(2-chloro-6-fluorophenyl)-2-((3-fluoro-4-((3R,5S)-3,4,5-trimethylpiperazin-1-yl)phenyl) amino)-8,9-dihydroimidazo[1,2-a]pyrimido[5,4-e]pyrimidin-5(6H)-one;
6-(2-chloro-6-fluorophenyl)-2-((3-chloro-4-((3R,5S)-3,4,5-trimethylpiperazin-1-yl)phenyl)-8,9-dihydroimidazo[1,2-a]pyrimido[5,4-e]pyrimidin-5(6H)-one;
6-(2-chloro-6-fluorophenyl)-2-((3-methyl-4-((3R,5S)-3,4,5-trimethylpiperazin-1-yl)phenyl) amino)-8,9-dihydroimidazo[1,2-a]pyrimido[5,4-e]pyrimidin-5(6H)-one;
6-(2-chloro-6-fluorophenyl)-2-((4-((3S,5R)-4-isopropyl-3,5-dimethylpiperazin-1-yl)-3-methylphenyl)amino)-8,9-dihydroimidazo[1,2-a]pyrimido[5,4-e]pyrimidin-5(6H)-one;
6-(2,6-dichlorophenyl)-2-((3-fluoro-4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-chloro-4-((3S,5R)-3,4,5-trimethylpiperazin-1-yl)phenyl) amino)-8,9-dihydroimidazo[1,2-a]pyrimido[5,4-e]pyrimidin-5(6H)-one;
6-(2,6-dichlorophenyl)-2-((3-methyl-4-((3S,5R)-3,4,5-trimethylpiperazin-1-yl)phenyl) amino)-8,9-dihydroimidazo[1,2-a]pyrimido[5,4-e]pyrimidin-5(6H)-one;
6-(2,6-dichlorophenyl)-2-((3-methyl-4-((3S,5R)-4-isopropyl-3,5-dimethylpiperazin-1-yl) phenyl) amino)-8,9-dihydroimidazo[1,2-a]pyrimido[5,4-e]pyrimidin-5(6H)-one;
6-(2,6-dichlorophenyl)-2-((3,5-dichloro-4-((3S,5R)-3,4,5-trimethylpiperazin-1-yl)phenyl) amino)-8,9-dihydroimidazo[1,2-a]pyrimido[5,4-e]pyrimidin-5(6H)-one; and
6-(2,6-dichlorophenyl)-2-((3-chloro-5-methyl-4-((3S,5R)-3,4,5-trimethylpiperazin-1-yl)phenyl) amino)-8,9-dihydroimidazo[1,2-a]pyrimido[5,4-e]pyrimidin-5(6H)-one.

In the preferred embodiments of Formula Ta, both of R₁ and R₂ are chloro.

In the preferred embodiments of Formula Ta, R₃ is halo, methyl or ethyl.

In the preferred embodiments of Formula Ta, R₇ is H, halo, methyl or methoxy.

In the preferred embodiments of Formula Ta, R₄ and R₆ each are independently methyl.

In the preferred embodiments of Formula Ta, R₅ is H, methyl or methyl-d3.

In the preferred embodiments of Formula Ta, both of R₁ and R₂ are chloro; R₃ is halo, methyl or ethyl; R₄ and R₆ each are independently methyl; R₅ is H, methyl or methyl-d3; R₇ is H. Preferably, R₄ and R₆ each are independently methyl; R₅ is H, methyl or methyl-d3; R₇ is H.

In the preferred embodiments of Formula Ta, both of R₁ and R₂ are chloro; R₃ is methyl or ethyl; R₄ and R₆ each are independently methyl; R₅ is methyl or methyl-d3; R₇ is halo, methyl or methoxy.

In the preferred embodiments of Formula I, compounds of Formula I have the structure as represented in the following Formula Ib:

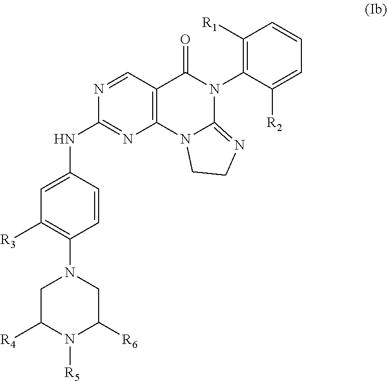

or stereoisomers, or pharmaceutically acceptable salts or prodrugs thereof, wherein:
R₁ and R₂ are independently halo; R₃ is C₁₋₄ alkyl; R₄ and R₆ are independently C₁₋₄ alkyl; R₅ are H or C₁₋₄ alkyl, and the alkyl group contains at least 3 deuterium (D).

In the preferred embodiments of Formula Ib, both of R₁ and R₂ are chloro.

In the preferred embodiments of Formula Ib, R₃ are methyl or ethyl.

In the preferred embodiments of Formula Ib, R₄ and R₆ each are independently methyl.

In the preferred embodiments of Formula Ib, R₅ is H or methyl-d3.

In the preferred embodiments of Formula Ib, both of R₁ and R₂ are chloro; R₃ is methyl or ethyl; R₄ and R₆ each are independently methyl; R₅ is H or methyl-d3.

In the preferred embodiments of Formula I, compounds of Formula I have the structure as represented in the following Formula Ic:

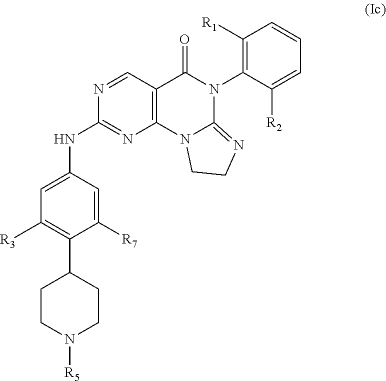

or stereoisomers, or pharmaceutically acceptable salts or prodrugs thereof.

In Formula Ic, R₁ and R₂ are independently halo; R₃ is halo, C₁₋₄ alkyl or C₁₋₄ alkoxy; R₅ is H or C₁₋₄ alkyl; R₇ is H, halo, C₁₋₄ alkyl or C₁₋₄ alkoxy.

In the preferred embodiments of Formula Ic, both of R₁ and R₂ are chloro.

In the preferred embodiments of Formula Ic, R₃ is halo, methyl or ethyl; more preferably, R₃ is F, Cl or methyl.

In the preferred embodiments of Formula Ic, R₇ is H, halo, methyl or ethyl; more preferably, R₇ is H, F, Cl or methyl.

In the preferred embodiments of Formula Ic, R₅ is C₁₋₄ alkyl; more preferably, R₅ is methyl or methyl-d3.

In the preferred embodiments of Formula Ic, both of R₁ and R₂ are chloro; R₃ is halo or C₁₋₄ alkyl; R₅ is C₁₋₄ alkyl; R₇ is H or halo.

In the preferred embodiments of Formula Ic, both of R₁ and R₂ are chloro; R₃ is halo or C₁₋₄ alkyl; R₅ is methyl or methyl-d3; R₇ is H, halo, methyl or ethyl.

Preferred compounds of Formula I include, without limitation:

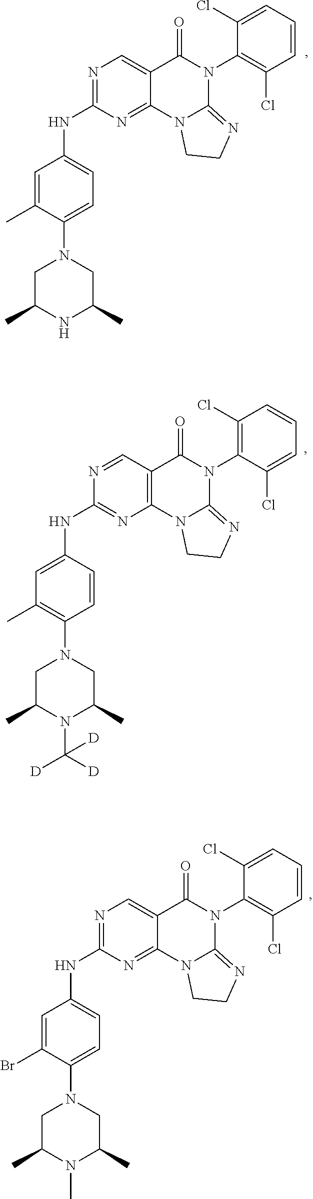 [SMILES: Cc1cc(Nc2ncc3c(n2)N2CCN=C2N(c2c(Cl)cccc2Cl)C3=O)ccc1N1C[C@@H](C)N[C@@H](C)C1 ; [2H]C([2H])([2H])N1[C@H](C)CN(c2ccc(Nc3ncc4c(n3)N3CCN=C3N(c3c(Cl)cccc3Cl)C4=O)cc2C)C[C@@H]1C ; C[C@@H]1CN(c2ccc(Nc3ncc4c(n3)N3CCN=C3N(c3c(Cl)cccc3Cl)C4=O)cc2Br)C[C@H](C)N1C]

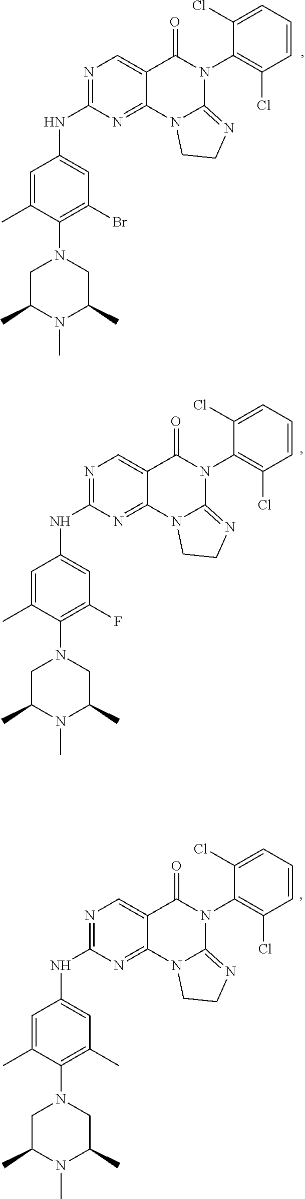 [SMILES: Cc1cc(Nc2ncc3c(n2)N2CCN=C2N(c2c(Cl)cccc2Cl)C3=O)cc(Br)c1N1C[C@@H](C)N(C)[C@@H](C)C1 ; Cc1cc(Nc2ncc3c(n2)N2CCN=C2N(c2c(Cl)cccc2Cl)C3=O)cc(F)c1N1C[C@@H](C)N(C)[C@@H](C)C1 ; Cc1cc(Nc2ncc3c(n2)N2CCN=C2N(c2c(Cl)cccc2Cl)C3=O)cc(C)c1N1C[C@@H](C)N(C)[C@@H](C)C1]

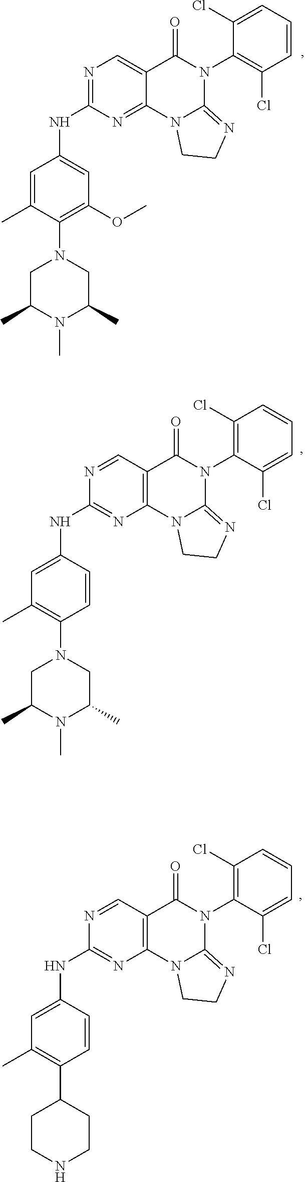 [SMILES: COc1cc(Nc2ncc3c(n2)N2CCN=C2N(c2c(Cl)cccc2Cl)C3=O)cc(C)c1N1C[C@@H](C)N(C)[C@@H](C)C1 ; Cc1cc(Nc2ncc3c(n2)N2CCN=C2N(c2c(Cl)cccc2Cl)C3=O)ccc1N1C[C@H](C)N(C)[C@@H](C)C1 ; Cc1cc(Nc2ncc3c(n2)N2CCN=C2N(c2c(Cl)cccc2Cl)C3=O)ccc1C1CCNCC1]

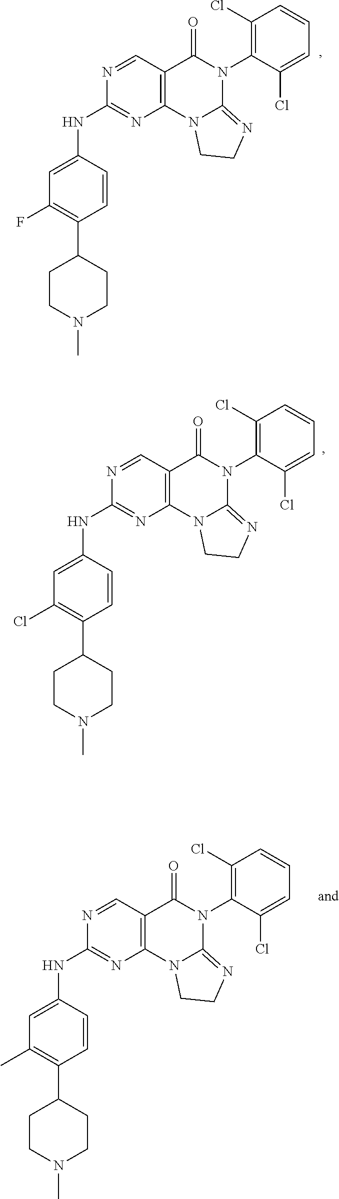 [SMILES: CN1CCC(c2ccc(Nc3ncc4c(n3)N3CCN=C3N(c3c(Cl)cccc3Cl)C4=O)cc2F)CC1 ; CN1CCC(c2ccc(Nc3ncc4c(n3)N3CCN=C3N(c3c(Cl)cccc3Cl)C4=O)cc2Cl)CC1 ; Cc1cc(Nc2ncc3c(n2)N2CCN=C2N(c2c(Cl)cccc2Cl)C3=O)ccc1C1CCN(C)CC1]

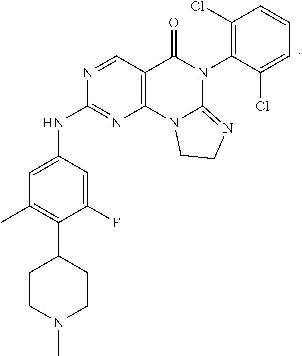 [SMILES: Cc1cc(Nc2ncc3c(n2)N2CCN=C2N(c2c(Cl)cccc2Cl)C3=O)cc(F)c1C1CCN(C)CC1]

Some of the compounds of the present disclosure may exist as stereoisomers including optical isomers. The disclosure includes all stereoisomers and the racemic mixtures of such stereoisomers as well as the individual enantiomers that may be separated according to methods that are well known to those of ordinary skill in the art.

Examples of pharmaceutically acceptable salts include inorganic and organic acid salts, such as hydrochloride, hydrobromide, phosphate, sulphate, citrate, lactate, tartrate, maleate, fumarate, mandelate and oxalate; and inorganic and organic base salts formed with bases, such as sodium hydroxy, tris(hydroxymethyl)aminomethane (TRIS, tromethamine) and N-methyl-glucamine.

Examples of prodrugs of the compounds of the disclosure include the simple esters of carboxylic acid-containing compounds (e.g., those obtained by condensation with a C₁-C₄ alcohol according to methods known in the art); esters of hydroxy containing compounds (e.g., those obtained by condensation with a C₁-C₄ carboxylic acid, C₃-C₆ diacid or anhydride thereof, such as succinic anhydride and fumaric anhydride according to methods known in the art); imines of amino containing compounds (e.g., those obtained by condensation with a C₁-C₄ aldehyde or ketone according to methods known in the art); carbamate of amino containing compounds, such as those described by Leu, et al., (J. Med. Chem. 42:3623-3628 (1999)) and Greenwald, et al., (J. Med. Chem. 42:3657-3667 (1999)); and acetals and ketals of alcohol-containing compounds (e.g., those obtained by condensation with chloromethyl methyl ether or chloromethyl ethyl ether according to methods known in the art).

The compounds of this disclosure may be prepared using methods known to those skilled in the art, or the novel methods of this disclosure. Specifically, the compounds of this disclosure with Formula I (including Formulae Ia, Ib and Ic) can be prepared as illustrated by the exemplary reaction in Scheme 1. Room temperature reaction of 6-(2,6-dichlorophenyl)-2-(methylthio)-8,9-dihydroimidazo[1,2-a]pyrimido[5,4-e]pyrimidin-5(6H)-one and 3-chloroperoxybenzoic acid in dichloromethane to produce 6-(2,6-dichlorophenyl)-2-(methylsulfinyl)-8,9-dihydroimidazo[1,2-a]pyrimido[5,4-e]pyrimidin-5(6H)-one and 6-(2,6-dichlorophenyl)-2-(methylsulfonyl)-8,9-dihydroimidazo[1,2-a]pyrimido[5,4-e]pyrimidin-5(6H)-one. Under the catalysis of trifluoroacetic acid, room temperature reaction of the mixture of 6-(2,6-dichlorophenyl)-2-(methylsulfinyl)-8,9-dihydroimidazo[1,2-a]pyrimido[5,4-e]pyrimidin-5(6H)-one and 6-(2,6-dichlorophenyl)-2-(methylsulfonyl)-8,9-dihydroimidazo[1,2-a]pyrimido[5,4-e]pyrimidin-5(6H)-one and tert-butyl (2S,6R)-4-(4-amino-2-methylphenyl)-2,6-dimethylpiperazine-1-carboxylate to produce tert-butyl (2S,6R)-4-(4-((6-(2,6-dichlorophenyl)-5-oxo-5,6,8,9-tetrahydroimidazo[1,2-a]pyrimido[5,4-e]pyrimidin-2-yl)amino)-2-methylphenyl)-2,6-dimethylpiperazine-1-carboxylate. Room temperature reaction of tert-butyl (2S,6R)-4-(4-((6-(2,6-dichlorophenyl)-5-oxo-5,6,8,9-tetrahydroimidazo[1,2-a]pyrimido[5,4-e]pyrimidin-2-yl)amino)-2-methylphenyl)-2,6-dimethylpiperazine-1-carboxylate and the methanol solution of hydrogen chloride in methanol to produce the target compound 6-(2,6-dichlorophenyl)-2-((4-((3S,5R)-3,5-dimethylpiperazin-1-yl)-3-methylphenyl)amino)-8,9-dihydroimidazo[1,2-a]pyrimido[5,4-e]pyrimidin-5(6H)-one.

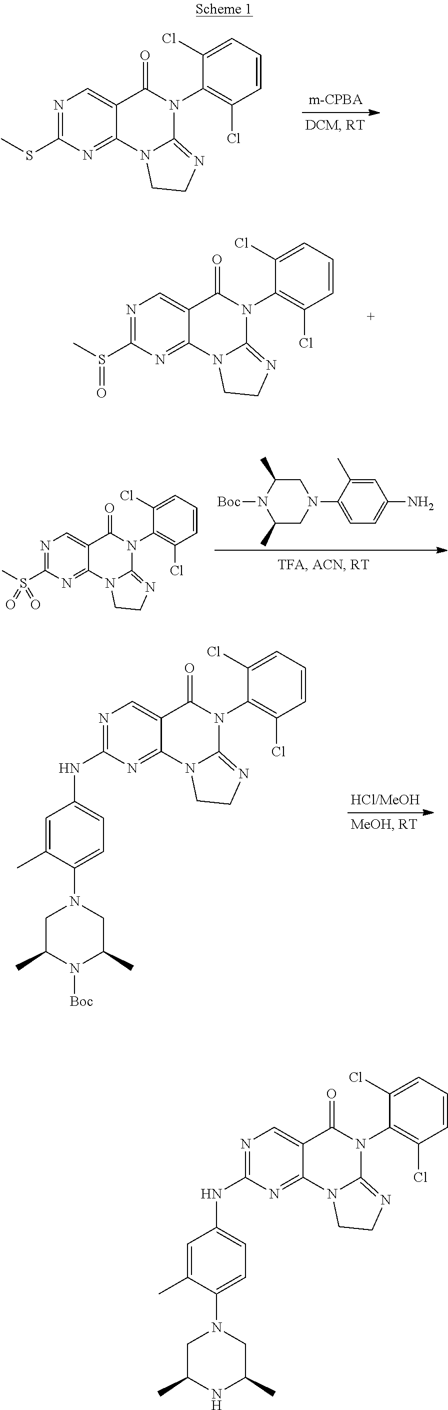

Other related compounds can be prepared using similar methods. For example, replacement of tert-butyl (2S,6R)-4-(4-amino-2-methylphenyl)-2,6-dimethylpiperazine-1-carboxylate with 4-((3S,5R)-3,5-dimethyl-4-(methyl-d3)piperazin-1-yl)-3-methylaniline produced the target compound 6-(2,6-dichlorophenyl)-2-((4-((3S,5R)-3,5-dimethyl-4-(methyl-d3)piperazin-1-yl)-3-methylphenyl)amino)-8,9-dihydroimidazo[1,2-a]pyrimido[5,4-e]pyrimidin-5(6H)-one. Replacement of (2S,6R)-4-(4-amino-2-methylphenyl)-2,6-dimethylpiperazine-1-carboxylate with 3-bromo-4-((3S,5R)-3,4,5-trimethylpiperazin-1-yl)aniline produced the target compound 2-((3-bromo-4-((3S,5R)-3,4,5-trimethylpiperazin-1-yl)phenyl)amino)-6-(2,6-dichlorophenyl)-8,9-dihydroimidazo[1,2-a]pyrimido[5,4-e]pyrimidin-5(6H)-one. Replacement of (2S,6R)-4-(4-amino-2-methylphenyl)-2,6-dimethylpiperazine-1-carboxylate with 3-fluoro-5-methyl-4-((3S,5R)-3,4,5-trimethylpiperazin-1-yl)aniline produced the target compound 6-(2,6-dichlorophenyl)-2-((3-fluoro-5-methyl-4-((3S,5R)-3,4,5-trimethylpiperazin-1-yl)phenyl)amino)-8,9-dihydroimidazo[1,2-a]pyrimido[5,4-e]pyrimidin-5(6H)-one. Replacement of (2S,6R)-4-(4-amino-2-methylphenyl)-2,6-dimethylpiperazine-1-carboxylate with 3-methyl-4-((3S,5S)-3,4,5-trimethylpiperazin-1-yl)aniline produced the target compound 6-(2,6-dichlorophenyl)-2-((3-methyl-4-((3S,5S)-3,4,5-trimethylpiperazin-1-yl)phenyl)amino)-8,9-dihydroimidazo[1,2-a]pyrimido[5,4-e]pyrimidin-5(6H)-one. Replacement of (2S,6R)-4-(4-amino-2-methylphenyl)-2,6-dimethylpiperazine-1-carboxylate with 3-methyl-4-(piperidin-4-yl)aniline produced the target compound 6-(2,6-dichlorophenyl)-2-((3-methyl-4-(piperidin-4-yl)phenyl)amino)-8,9-dihydroimidazo[1,2-a]pyrimido[5,4-e]pyrimidin-5(6H)-one. Replacement of (2S,6R)-4-(4-amino-2-methylphenyl)-2,6-dimethylpiperazine-1-carboxylate with 3-methyl-4-(1-methylpiperidin-4-yl)aniline produced the target compound 6-(2,6-dichlorophenyl)-2-((3-methyl-4-(1-methylpiperidin-4-yl)phenyl)amino)-8,9-dihydroimidazo[1,2-a]pyrimido[5,4-e]pyrimidin-5(6H)-one. Replacement of (2S,6R)-4-(4-amino-2-methylphenyl)-2,6-dimethylpiperazine-1-carboxylate with 3-fluoro-5-methyl-4-(1-methylpiperidin-4-yl)aniline produced the target compound 6-(2,6-dichlorophenyl)-2-((3-fluoro-5-methyl-4-(1-methylpiperidin-4-yl)phenyl)amino)-8,9-dihydroimidazo[1,2-a]pyrimido[5,4-e]pyrimidin-5(6H)-one.

An important aspect of the present disclosure is the discovery that compounds having Formula I (including Formulae Ia, Ib and Ic) are kinase inhibitors, especially Wee1 kinase inhibitors with good activity. Therefore, these compounds are useful for the treatment of Wee1-related diseases, Wee1-mediated diseases, such as cancer. Herein, Wee 1-mediated diseases refer to diseases for which inhibition of Wee 1 activity is required for treatment or prevention.

The present disclosure includes a therapeutic method comprising administering to a mammal an effective amount of a compound of Formula I (including Formulae Ia, Ib and Ic) or stereoisomers, or a pharmaceutically acceptable salt or prodrug thereof, wherein said therapeutic method is useful for the treatment of diseases related with kinase, especially Wee1 kinase, such as cancer. Such diseases that can be treated or prevented by the method or pharmaceutical composition of the present disclosure include, but are not limited to, liver cancer, melanoma, Hodgkin's disease, non-Hodgkin's lymphoma, acute lymphocytic leukemia, chronic lymphocytic leukemia, multiple myeloma, neuroblastoma, breast carcinoma, ovarian carcinoma, lung carcinoma, Wilms' tumor, cervical carcinoma, testicular carcinoma, soft-tissue sarcoma, primary macroglobulinemia, bladder carcinoma, chronic granulocytic leukemia, primary brain carcinoma, malignant melanoma, small-cell lung carcinoma, stomach carcinoma, colon carcinoma, malignant pancreatic insulinoma, malignant carcinoid carcinoma, choriocarcinoma, mycosis fungoides, head and neck carcinoma, osteogenic sarcoma, pancreatic carcinoma, acute granulocytic leukemia, hairy cell leukemia, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, adrenal cortex carcinoma, skin cancer, and prostatic carcinoma.

Compounds of the present disclosure also are useful for the treatment or prevention of other diseases due to abnormal kinase activity, especially Wee1, such as neurology or neuropsychiatric diseases or conditions, such as depression.

In practicing the therapeutic methods, effective amounts of compositions containing therapeutically effective concentrations of the compounds of Formula I (including Formulae Ta, Tb and Ic) or stereoisomers, or a pharmaceutically acceptable salt or prodrug thereof, which was formulated for oral, intravenous, local or topical application, for the treatment of cancer and other diseases, are administered to an individual exhibiting the symptoms of one or more of these disorders. The amounts are effective to ameliorate or eliminate one or more symptoms of the disorders. An effective amount of a compound for treating a particular disease is an amount that is sufficient to ameliorate, or in some manner reduce, the symptoms associated with the disease. Such amount may be administered as a single dosage or may be administered according to an effective regimen. The amount may cure the disease but, typically, is administered in order to ameliorate the symptoms of the disease. Typically, repeated administration is required to achieve the desired amelioration of symptom.

In another embodiment, a pharmaceutical composition comprising a compound of Formula I (including Formulae Ta, Tb and Ic) or stereoisomers, or a pharmaceutically acceptable salt or prodrug thereof, which functions as kinase inhibitor, in combination with a pharmaceutically acceptable vehicle, is provided.

Another embodiment of the present disclosure is directed to a composition effective to treat cancer comprising a compound of Formula I (including Formulae Ta, Tb and Ic) or stereoisomers, or a pharmaceutically acceptable salt or prodrug thereof, which functions as a kinase inhibitor, in combination with at least one known anticancer agent or a pharmaceutically acceptable salt thereof. In particular, the compound herein can be combined with other anticancer drugs related to the mechanism of DNA damage and repair, including PARP inhibitors Olaparib, Niraprib, Rucaparib, Talazoparib and Senaparib; HDAC inhibitors Volinota, Romididesin, Papiseta and Bailesta; and so on. And the compound herein can be combined with other anticancer drugs related to cell division detection sites, including Chk1/2 inhibitors, CDK4/6 inhibitors such as Paposinib, ATM/ATR inhibitors, and so on. Other examples of known anticancer agents which may be used for combination therapy include, but not are limited to alkylating agents, such as busulfan, melphalan, chlorambucil, cyclophosphamide, ifosfamide, temozolomide, bendamustine, cis-platin, mitomycin C, bleomycin, and carboplatin; topoisomerase I inhibitors, such as camptothecin, irinotecan, and topotecan; topoisomerase II inhibitors, such as doxorubicin, epirubicin, aclarubicin, mitoxantrone, elliptinium and etoposide; RNA/DNA antimetabolites, such as 5-azacytidine, gemcitabine, 5-fluorouracil and methotrexate; DNA antimetabolites, such as 5-fluoro-2′-deoxy-uridine, fludarabine, nelarabine, ara-C, pralatrexate, pemetrexed, hydroxyurea and thioguanine; antimitotic agents, such as colchicine, vinblastine, vincristine, vinorelbine, paclitaxel, ixabepilone, cabazitaxel and docetaxel; antibodies such as campath, panitumumab, metazotuzumab, navuzumab, pymzumab, remoluzumab, bevacizumab, partuzumab, trastuzumab, cetuximab, obinutuzumab, olfactuzumab, rituximab, alemtuzumab, tiemuzumab, toximab, bentuximab, daremuzumab, errotuzumab, T-DM1, ofatumumab, dinutuximab, blinatumomab, ipilimma, avastin, trastuzumab and rituximab; kinase inhibitors such as imatinib, gefitinib, erlotinib, osimertinib, afatinib, ceritinib, aletinib, crizotinib, erlotinib, lapatinib, sorafenib, regorafenib, vemurafenib, dabrafenib, aflibercept, sunitinib, nilotinib, dasatinib, bosutinib, pratinib, ibrutinib, cabozatinib, lenvatinib, vandetanib, trametinib, cobimetinib, axitinib, temsirolimus, idelalisib, pazopanib, temsirolimus and everolimus. Other known anticancer agents which may be used for combination therapy include tamoxifen, letrozole, fulvestrant, mitoguazone, octreotide, retinoic acid, arsenic trioxide, zoledronic acid, bortezomib, carfazomide, ixazomib, erivedge, sonidegib, denosumab, thalidomide, lenalidomide, venetoclax, aldesleukin (recombinant human interleukin-2) and sipueucel-T (prostate cancer therapeutic vaccine).

In practicing the methods of the present disclosure, the compound of the disclosure may be administered together with at least one known anticancer agent as part of a unitary pharmaceutical composition. Alternatively, the compound of the disclosure may be administered apart from at least one known anticancer agent. In one embodiment, the compound of the disclosure and at least one known anticancer agent are administered substantially simultaneously, i.e. the compounds are administered at the same time or one after the other, so long as the compounds reach therapeutic levels in the blood at the same time. In another embodiment, the compound of the disclosure and at least one known anticancer agent are administered according to their individual dose schedule, so long as the compounds reach therapeutic levels in the blood.

Another embodiment of the present disclosure is directed to a composition effective to inhibit neoplasia comprising a bioconjugate of a compound described herein, which functions as a kinase inhibitor, in bioconjugation with at least one known therapeutically useful antibody, such as trastuzumab or rituximab, growth factors, such as DGF, NGF; cytokines, such as IL-2, IL-4, or any molecule that binds to the cell surface. The antibodies and other molecules will deliver a compound described herein to its targets and make it an effective anticancer agent. The bioconjugates could also enhance the anticancer effect of the therapeutically useful antibodies, such as trastuzumab or rituximab.

Similarly, another embodiment of the present disclosure is directed to a composition effective to inhibit neoplasia comprising a compound of Formula I (including Formulae Ia, lb and Ic), or its pharmaceutically acceptable salt or prodrug, which functions as a kinase inhibitor, in combination with radiation therapy. In this embodiment, the compound of the disclosure may be administered at the same time as the radiation therapy is administered or at a different time.

Yet another embodiment of the present disclosure is directed to a composition effective for post-surgical treatment of cancer, comprising a compound of Formula I (including Formulae Ia, Ib and Ic) or stereoisomers, or a pharmaceutically acceptable salt or prodrug thereof, which functions as a kinase inhibitor. The disclosure also relates to a method of treating cancer by surgically removing the tumor and then treating the mammal with one of the pharmaceutical compositions described herein.

Pharmaceutical compositions within the scope of this disclosure include all compositions wherein the compounds of the present disclosure are contained in an amount that is effective to achieve its intended purpose. While individual needs vary, determination of optimal ranges of effective amounts of each component is within the skill of the art. Typically, the compounds may be administered to mammals, orally at a dose of from about 0.0025 to 50 mg/kg of body weight, per day, or an equivalent amount of the pharmaceutically acceptable salt thereof, to a mammal being treated. Preferably, from approximately 0.01 to approximately 10 mg/kg of body weight is orally administered. If a known anticancer agent is also administered, it is administered in an amount that is effective to achieve its intended purpose. The optimal amounts of such known anticancer agents effective for cancer are well known to those skilled in the art.

The unit oral dose may comprise from approximately 0.01 to approximately 50 mg, preferably approximately 0.1 to approximately 10 mg of the compound of the disclosure. The unit dose may be administered one or more times daily, as one or more tablets, each containing from approximately 0.1 to approximately 50 mg, conveniently approximately 0.25 to 10 mg of the compound or its solvates.

In a topical formulation, the compound may be present at a concentration of approximately 0.01 to 100 mg per gram of carrier.

In addition to administering the compound as a raw chemical, the compounds of the disclosure may be administered as part of a pharmaceutical preparation containing suitable pharmaceutically acceptable carriers comprising excipients and auxiliaries, which facilitate processing of the compounds into preparations that may be used pharmaceutically. Preferably, the preparations, particularly those preparations which may be administered orally and that may be used for the preferred type of administration, such as tablets, dragees, and capsules, as well as suitable solutions for administration by injection or orally, contain from approximately 0.01 to 99 percent, preferably from approximately 0.25 to 75 percent of active compound(s), together with the excipient.

Also included within the scope of the present disclosure are the non-toxic pharmaceutically acceptable salts of the compounds of the present disclosure. Acid addition salts are formed by mixing a solution of the compounds of the present disclosure with a solution of a pharmaceutically acceptable non-toxic acid, such as hydrochloric acid, fumaric acid, maleic acid, succinic acid, acetic acid, citric acid, tartaric acid, carbonic acid, phosphoric acid, oxalic acid, and the like. Base addition salts are formed by mixing a solution of the compounds of the present disclosure with a solution of a pharmaceutically acceptable non-toxic base, such as sodium hydroxide, potassium hydroxide, choline hydroxide, sodium carbonate, tris(hydroxymethyl)aminomethane (TRIS), N-methyl-glucamine and the like.

The pharmaceutical compositions of the disclosure may be administered to any mammal, so long as they may experience the therapeutic effects of the compounds of the disclosure. Foremost among such mammals are humans and veterinary animals, although the disclosure is not intended to be so limited.

The pharmaceutical compositions of the present disclosure may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present disclosure are manufactured in a manner, which is itself known, e.g., by means of conventional mixing, granulating, dragee-making, dissolving, or lyophilizing processes. Thus, pharmaceutical preparations for oral use may be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular: fillers, such as saccharides, e.g. lactose or sucrose, mannitol or sorbitol; cellulose preparations and/or calcium phosphates, e.g. tricalcium phosphate or calcium hydrogen phosphate; as well as binders, such as starch paste, using, e.g., maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added, such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, e.g., silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations, such as acetylcellulose phthalate or hydroxypropylmethyl-cellulose phthalate, are used. Dye stuffs or pigments may be added to the tablets or dragee coatings, e.g., for identification or in order to characterize combinations of active compound doses.

Other pharmaceutical preparations, which may be used orally, include push-fit capsules made of gelatin, as well as soft, sealed capsules made of gelatin and a plasticizer, such as glycerol or sorbitol. The push-fit capsules may contain the active compounds in the form of: granules, which may be mixed with fillers, such as lactose; binders, such as starches; and/or lubricants, such as talc or magnesium stearate and, optionally, stabilizers. In soft capsules, the active compounds are preferably dissolved or suspended in suitable liquids, such as fatty oils, or liquid paraffin. In addition, stabilizers may be added.

Suitable formulations for parenteral administration include aqueous solutions of the active compounds, e.g., aqueous solutions and alkaline solutions of water-soluble salts. In addition, suspensions of the active compounds as appropriate oily injection suspensions may be administered. Suitable lipophilic solvents or vehicles include fatty oils, e.g., sesame oil, or synthetic fatty acid esters, e.g., ethyl oleate or triglycerides or polyethylene glycol-400, or cremophor, or cyclodextrins. Aqueous injection suspensions may contain substances which increase the viscosity of the suspension, e.g., sodium carboxymethyl cellulose, sorbitol, and/or dextran. Optionally, the suspension may also contain stabilizers.

In accordance with one aspect of the present disclosure, compounds of the disclosure are employed in topical and parenteral formulations and are used for the treatment of skin cancer.

The topical compositions of this disclosure are formulated preferably as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The preferred carriers are those in which the active ingredient is soluble. Emulsifiers, stabilizers, humectants and antioxidants may also be included, as well as agents imparting color or fragrance, if desired. Additionally, transdermal penetration enhancers may be employed in these topical formulations. Examples of such enhancers are found in U.S. Pat. Nos. 3,989,816 and 4,444,762.

Creams are preferably formulated from a mixture of mineral oil, self-emulsifying beeswax and water in which mixture of the active ingredient, dissolved in a small amount of an oil, such as almond oil, is admixed. A typical example of such a cream is one which includes approximately 40 parts water, approximately 20 parts beeswax, approximately 40 parts mineral oil and approximately 1 part almond oil.

Ointments may be formulated by mixing a solution of the active ingredient in a vegetable oil, such as almond oil, with warm soft paraffin and allowing the mixture to cool. A typical example of such an ointment is one which includes approximately 30% almond oil and approximately 70% white soft paraffin by weight.

The present disclosure also includes the use of the compounds of the subject disclosure in the manufacture of a medicament for treating a clinical condition responsive to the inhibition of kinase (especially Wee1) activity. The medicament may include the pharmaceutical compositions as described above.

The following examples are illustrative, but not limiting, of the method and compositions of the present disclosure. Other suitable modifications and adaptations of the variety of conditions and parameters normally encountered in clinical therapy and which are obvious to those skilled in the art are within the spirit and scope of the disclosure.

### EXAMPLES

### General Remarks

All reagents were of commercial quality. Solvents were dried and purified by standard methods. Mass spectrum analyses were recorded on a Platform II (Agilent 6110) quadrupole mass spectrometer fitted with an electrospray interface. ¹H NMR spectra was recorded at 400 MHz, on a Brücker Ascend 400 apparatus. Chemical shifts were recorded as parts per million (ppm) downfield from TMS (0.00 ppm), and J coupling constants were reported in hertz (Hz).

### Example 1

### 6-(2,6-dichlorophenyl)-2-((4-((3S,5R)-3,5-dimethylpiperazin-1-yl)-3-methylphenyl)amino)-8,9-dihydroimidazo[1,2-a]pyrimido[5,4-e]pyrimidin-5(6H)-one

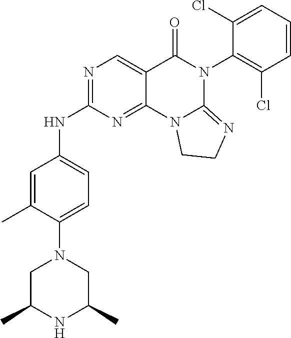 [SMILES: Cc1cc(Nc2ncc3c(n2)N2CCN=C2N(c2c(Cl)cccc2Cl)C3=O)ccc1N1C[C@@H](C)N[C@@H](C)C1]

### A) Preparation of tert-butyl (2S,6R)-4-(4-amino-2-methylphenyl)-2,6-dimethylpiperazine-1-carboxylate

a) Preparation of (3S,5R)-3,5-dimethyl-1-(2-methyl-4-nitrophenyl)piperazine: To a solution of 1-fluoro-2-methyl-4-nitrobenzene (25 g, 161.16 mmol) in DMSO (500 mL) was added K₂CO₃ (66.82 g, 483.48 mmol) and (2S,6R)-2,6-dimethylpiperazine (21.53 g, 188.56 mmol). The mixture was stirred at 100° C. for 6 h. The reaction mixture was diluted with H₂O (2.5 L) and extracted with EA (1 L×3). The combined organic layers were washed with brine (1 L×2), dried over Na₂SO₄, filtered and concentrated under reduced pressure to give the target compound (38 g, brown oil, 94.58% yield).

b) Preparation of tert-butyl (2S,6R)-2,6-dimethyl-4-(2-methyl-4-nitrophenyl)piperazine-1-carboxylate: To a solution of (3S,5R)-3,5-dimethyl-1-(2-methyl-4-nitrophenyl)piperazine (38 g, 152.42 mmol) in DCM (380 mL) was added DIPEA (29.55 g, 228.63 mmol, 39.82 mL) and Boc₂O (39.92 g, 182.91 mmol, 42.02 mL). The mixture was stirred at 25° C. for 24 h. LCMS showed 19.4% of (3S,5R)-3,5-dimethyl-1-(2-methyl-4-nitrophenyl)piperazine was remained. To a solution of the mixture was added DIPEA (15.76 g, 121.94 mmol, 21.24 mL) and Boc₂O (16.63 g, 76.21 mmol, 17.51 mL). The mixture was stirred at 25° C. for 12 h. The reaction mixture was concentrated under reduced pressure to give a residue. The residue was purified by column chromatography to give the target compound (45 g, 128.78 mmol, yellow solid, 84.49% yield). LC-MS (ESI): m/z (M-55)⁺ 294.2. ¹H NMR (400 MHz, CDCl₃): δ 8.08-8.05 (m, 2H), 7.06-7.04 (m, 1H), 4.29 (t, J 5.2 Hz, 2H), 3.06 (d, J 11.6 Hz, 1H), 2.88 (dd, J 4.0, 11.6 Hz, 2H), 2.48 (s, 3H), 1.51 (s, 9H), 1.46 (s, 3H), 1.44 (s, 3H).

c) Preparation of tert-butyl (2S,6R)-4-(4-amino-2-methylphenyl)-2,6-dimethylpiperazine-1-carboxylate: To a solution of tert-butyl (2S,6R)-2,6-dimethyl-4-(2-methyl-4-nitrophenyl)piperazine-1-carboxylate (25 g, 71.55 mmol) in MeOH (250 mL) was added Pd/C (5 g, 10% purity) under H₂ (25 psi) atmosphere. The mixture was stirred at 15° C. for 12 h. The reaction mixture was filtered and concentrated under reduced pressure to give the target compound (22.5 g, 70.44 mmol, brown oil, 98.45% yield). LC-MS (ESI): m/z (M+1)+320.0.

### B) Preparation of 6-(2,6-dichlorophenyl)-2-((4-((3S,5R)-3,5-dimethylpiperazin-1-yl)-3-methylphenyl)amino)-8,9-dihydroimidazo[1,2-a]pyrimido[5,4-e]pyrimidin-5(6H)-one

a) Preparation of 6-(2,6-dichlorophenyl)-2-(methylsulfinyl)-8,9-dihydroimidazo[1,2-a]pyrimido[5,4-e]pyrimidin-5(6H)-one and 6-(2,6-dichlorophenyl)-2-(methylsulfonyl)-8,9-dihydroimidazo[1,2-a]pyrimido[5,4-e]pyrimidin-5(6H)-one: To a solution of 6-(2,6-dichlorophenyl)-2-(methylthio)-8,9-dihydroimidazo[1,2-a]pyrimido[5,4-e]pyrimidin-5(6H)-one (24.7 g, 64.96 mmol) in DCM (250 mL) was added m-CPBA (28.02 g, 129.91 mmol, 80% purity) at 0° C. The mixture was stirred at 25° C. for 2 h. The reaction was quenched with H₂O (100 mL) at 0° C. The reaction mixture was diluted with DCM (100 mL) and washed with H₂O (100 mL×2), NaHCO₃ solution (100 mL×2), Na₂SO₃ solution (5 wt %, 100 mL×2) and brine (100 mL×2), dried over Na₂SO₄, filtered and concentrated under reduced pressure to give a residue. The residue was washed with MTBE (40 mL) to give a mixture of the target compound (19.2 g, yellow solid). LC-MS (ESI): m/z (M+1)+395.8; (M+1)+411.8.

b) Preparation of tert-butyl (2S,6R)-4-(4-((6-(2,6-dichlorophenyl)-5-oxo-5,6,8,9-tetrahydroimidazo[1,2-a]pyrimido[5,4-e]pyrimidin-2-yl)amino)-2-methylphenyl)-2,6-dimethylpiperazine-1-carboxylate: To a solution of tert-butyl (2S,6R)-4-(4-amino-2-methylphenyl)-2,6-dimethylpiperazine-1-carboxylate (15.3 g, 47.90 mmol) in MeCN (153 mL) was added TFA (148.94 mg, 1.31 mmol, 96.72 μL) and 6-(2,6-dichlorophenyl)-2-(methylsulfinyl)-8,9-dihydroimidazo[1,2-a]pyrimido[5,4-e]pyrimidin-5(6H)-one and 6-(2,6-dichlorophenyl)-2-(methylsulfonyl)-8,9-dihydroimidazo[1,2-a]pyrimido[5,4-e]pyrimidin-5(6H)-one (17.28 g). The mixture was stirred at 25° C. for 2 h. The reaction mixture was filtered and the filter cake was dried in vacuo, the cake was washed with MeCN (200 mL) and MeOH (200 mL) to give the target compound (15 g, 22.84 mmol, yellow solid, 52.45% yield). LC-MS (ESI): m/z (M+1)+651.2. ¹H NMR (400 MHz, CDCl₃): δ 8.83 (s, 1H), 7.49-7.45 (m, 4H), 7.39-7.37 (m, 1H), 7.05-7.03 (d, J=8.4 Hz, 1H), 4.24 (t, J=4.8 Hz, 4H), 4.03 (t, J=9.2 Hz, 2H), 2.93-2.82 (m, 4H), 2.44 (s, 3H), 3.12 (s, 9H), 1.46 (s, 3H), 1.44 (s, 3H).

c) Preparation of 6-(2,6-dichlorophenyl)-2-((4-((3S,5R)-3,5-dimethylpiperazin-1-yl)-3-methylphenyl)amino)-8,9-dihydroimidazo[1,2-a]pyrimido[5,4-e]pyrimidin-5(6H)-one: To a solution of tert-butyl (2S,6R)-4-(4-((6-(2,6-dichlorophenyl)-5-oxo-5,6,8,9-tetrahydroimidazo[1,2-a]pyrimido[5,4-e]pyrimidin-2-yl)amino)-2-methylphenyl)-2,6-dimethylpiperazine-1-carboxylate (8.24 g, 12.54 mmol) in MeOH (63 mL) was added HCl/MeOH (4 M, 62.72 mL). The mixture was stirred at 25° C. for 24 h. The reaction mixture was concentrated under reduced pressure, and the residue was dissolved in H₂O (200 mL), and the mixture was adjusted to pH 8 with NaHCO₃ solution. The mixture was filtered and the filter cake was washed with H₂O (50 mL), and the cake was dried in vacuo. The filter cake was washed with MeCN (40 mL), the mixture was filtered and the filter cake was dried in vacuo. The product was suspended in the mixed solvent of water (100 mL) and methanol (20 mL), and lyophilized to give the target compound (6.2 g, 11.10 mmol, yellow solid, 88.50% yield). LC-MS (ESI): m/z (M+1)+551.2. ¹H NMR (400 MHz, DMSO-d6): δ 10.31-10.24 (m, 1H), 8.67 (s, 1H), 7.68-7.45 (m, 5H), 6.98 (d, J=8.4 Hz, 1H), 4.17 (d, J=7.2 Hz, 2H), 3.82 (t, J=9.2 Hz, 2H), 3.06 (s, 2H), 2.94 (d, J=10.8 Hz, 2H), 2.33-2.25 (m, 5H), 1.07 (s, 3H), 1.05 (s, 3H).

### Example 2

### 6-(2,6-dichlorophenyl)-2-((4-((3S,5R)-3,5-dimethyl-4-(methyl-d3)piperazin-1-yl)-3-methylphenyl)amino)-8,9-dihydroimidazo[1,2-a]pyrimido[5,4-e]pyrimidin-5(6H)-one

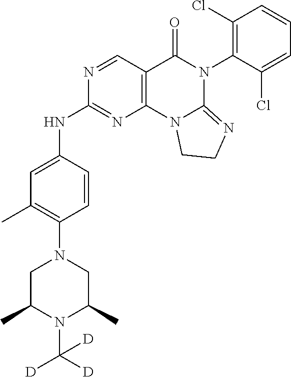 [SMILES: [2H]C([2H])([2H])N1[C@H](C)CN(c2ccc(Nc3ncc4c(n3)N3CCN=C3N(c3c(Cl)cccc3Cl)C4=O)cc2C)C[C@@H]1C]

a) Preparation of (2S,6R)-2,6-dimethyl-1-(methyl-d3)-4-(2-methyl-4-nitrophenyl)piperazine: To a solution of (3S,5R)-3,5-dimethyl-1-(2-methyl-4-nitro-phenyl)piperazine (2 g, 8.02 mmol) in DMF (15 mL) was added NaH (385.03 mg, 9.63 mmol, 60% purity), the mixture was stirred at 0° C. for 25 min, and trideuterio(iodo)methane (1.16 g, 8.02 mmol, 499.09 μL) was added. The mixture was stirred at 0° C. for 2 hrs. The reaction mixture was quenched by addition NaHCO₃ solution (30 mL) at 0° C., and then extracted with EA (50 mL×3). The combined organic layers were dried over Na₂SO₄, filtered and concentrated under reduced pressure to give the target compound crude (1.5 g, yellow green solid). LC-MS (ESI): m/z (M+1)+267.1. ¹H NMR (400 MHz, CDCl₃): δ 8.04-8.01 (m, 2H), 6.96 (d, J=12.0 Hz, 1H), 3.10 (d, J=12 Hz, 2H), 2.65 (t, J=12 Hz, 2H), 2.45-2.43 (m, 2H), 2.36 (s, 3H), 1.16-1.15 (d, J=4.0 Hz, 6H).

b) Preparation of 4-((3S,5R)-3,5-dimethyl-4-(methyl-d3)piperazin-1-yl)-3-methylaniline: To a solution of (2S,6R)-2,6-dimethyl-4-(2-methyl-4-nitro-phenyl)-1-(trideuteriomethyl)piperazine (1.5 g, 5.63 mmol) in MeOH (5 mL) was added Pd/C (281.58 umol, 10% purity) under N2 atmosphere. The suspension was degassed and purged with H₂ for many times. The mixture was stirred under H₂ (15 psi) at 25° C. for 12 hr. The reaction mixture was filtered and concentrated under reduced pressure to give the target compound crude (1.3 g, black solid). LC-MS (ESI): m/z (M+1)+237.1.

c) Preparation of 6-(2,6-dichlorophenyl)-2-((4-((3S,5R)-3,5-dimethyl-4-(methyl-d3)piperazin-1-yl)-3-methylphenyl)amino)-8,9-dihydroimidazo[1,2-a]pyrimido[5,4-e]pyrimidin-5(6H)-one: To a solution of 4-((3S,5R)-3,5-dimethyl-4-(methyl-d3)piperazin-1-yl)-3-methylaniline (459.32 mg, 1.94 mmol) in MeCN (5 mL) was added TFA (20.14 mg, 0.177 mmol, 13.08 μL), and the mixture of 6-(2,6-dichlorophenyl)-2-(methylsulfinyl)-8,9-dihydroimidazo[1,2-a]pyrimido[5,4-e]pyrimidin-5(6H)-one and 6-(2,6-dichlorophenyl)-2-(methylsulfonyl)-8,9-dihydroimidazo[1,2-a]pyrimido[5,4-e]pyrimidin-5(6H)-one (700 mg, crude). The mixture was stirred at 25° C. for 2 hrs. The reaction mixture was filtered and concentrated under reduced pressure to give a residue. The residue purified by reversed-phase HPLC to give the target compound (56.89 mg, yellow solid, 5.66 yield). LC-MS (ESI): m/z (M+1)(568.0. ¹H NMR (400 MHz, CDCl₃): δ 8.81 (s, 1H), 7.49 (d, J=3.8 Hz, 3H), 7.41-7.34 (m, 3H), 7.02 (d, J=4.2 Hz, 1H), 4.25-4.21 (1, 2H), 4.02 (t, J=8.0 Hz, 2H), 2.95 (d, J=6.0 Hz 2H), 2.62 (t, J=6.0 Hz, 2H), 2.46-2.41 (m, 2H), 2.34 (s, 6H), 1.15 (d, J=6.4 Hz, 6H).

The following compounds of Examples 3-13 were prepared using a synthesis process similar to that described in Example 1 or Example 2.

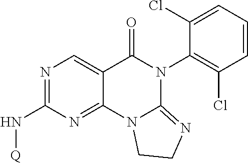

| Example | Q | LC-MS (ESI) | ¹H NMR (400 MHz) |
|---|---|---|---|
| 3 | 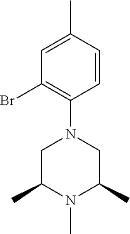 | (M/2 + 1) ⁺ 316.1 | DMSO-d₆: δ 8.71 (s, 1H), 8.13 (s, 1H), 7.80-7.77 (m, 1H), 7.68 (d, J = 7.6 Hz, 2H), 7.57-7.53 (m, 1H), 7.15 (d, J = 8.4 Hz, 1H), 4.19 (t, J = 8.4 Hz, 2H), 3.84 (t, J = 8.4 Hz, 2H), 3.20-3.08 (m, 2H), 2.81-2.52 (m, 7H), 1.12-1.07 (s, 6H). |
| 4 | 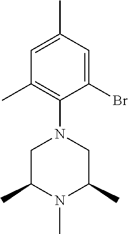 | (M + 1) ⁺ 645.10 | CDCl₃: δ 8.83 (s, 1H), 8.06-8.00 (m, 1H), 7.50- 7.48 (m, 2H), 7.39-7.34 (m, 2H), 7.20-7.15 (m, 1H), 4.26 (t, J = 8.8 Hz, 2H), 4.07 (t, J = 8.8 Hz, 2H), 3.53-3.47 (m, 1H), 3.17-3.12 (m, 1H), 2.91- 2.85 (m, 1H), 2.68-2.66 (m, 1H), 2.57-2.53 (m, 1H), 2.38-2.36 (m, 7H), 1.13 (d, J = 6 Hz, 6H). |
| 5 | 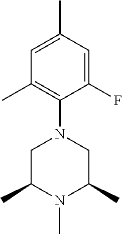 | (M + 1) ⁺ 583.1 | CDCl₃: δ 8.83 (s, 1H), 7.63-7.62 (m, 1H), 7.49 (d, J = 4.0 Hz, 2H), 7.39-7.35 (m, 2H), 7.99 (d, J = 4.0 Hz, 1H), 4.27-4.21 (m, 2H), 4.04 (t, J = 8.0 Hz, 2H), 3.13-3.07 (m, 2H), 2.81-2.77 (m, 2H), 2.43- 2.41 (m, 2H), 2.34 (s, 6H), 1.12 (t, J = 6.4 Hz, 6H). |
| 6 | 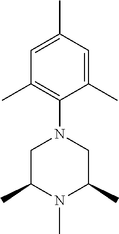 | (M + 1) ⁺ 579.0 | CDCl₃: δ 8.82 (s, 1H), 7.50-7.38 (m, 2H), 7.36- 7.34 (m, 3H), 4.23 (t, J = 8.0 Hz, 2H), 4.03 (t, J = 8.0 Hz, 1H), 3.15 (t, J = 11.2 Hz, 2H), 2.79-2.77 (m, 2H), 2.48-2.35 (m, 11H), 1.13 (d, J = 6.4 Hz, 6H). |
| 7 | 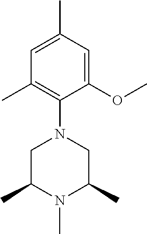 | (M + 1) ⁺ 595.3 | CDCl₃: δ 8.83 (s, 1H), 7.50 (t, J = 7.6 Hz, 2H), 7.36 (t, J = 8.0 Hz, 2H), 6.89 (d, J = 2.4 Hz, 1H), 4.23 (t, J = 8.4 Hz, 2H), 4.05-4.01 (m, 2H), 3.83 (s, 3H), 3.27 (t, J = 10.8 Hz, 2H), 2.66 (d, J = 10.8 Hz, 2H), 2.33-2.30 (m, 8H), 1.11 (d, J = 6.0 Hz, 6H). |
| 8 | 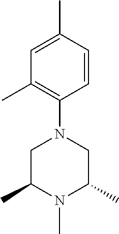 | (M + 1)⁺ 565.2 | DMSO-d₆: δ 10.30 (brs, 1H), 8.67 (s, 1H), 7.68- 7.51 (m, 5H), 6.98 (d, J = 8.4 Hz, 1H), 4.18-4.16 (m, 2H), 3.82 (t, J = 8.8 Hz, 2H), 2.85-2.80 (m, 4H), 2.58-2.57 (m, 2H), 2.28 (s, 3H), 2.25 (s, 3H), 1.09 (d, J = 6.0 Hz, 6H). |
| 9 | 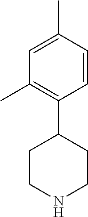 | (M + 1)⁺ 522.1 | CDCl₃: δ 8.83 (s, 1H), 7.53-7.48 (m, 3H), 7.42 (s, 1H), 7.38-7.34 (m, 1H), 7.27-7.24 (m, 1H), 4.27- 4.23 (m, 2H),4.03 (t, J = 8.0 Hz, 2H), 3.22 (d, J = 6.0 Hz, 2H), 2.83-2.76 (m, 3H), 3.38 (s, 3H), 1.79- 1.63 (m, 2H), 1.70-1.64 (m, 2H). |
| 10 | 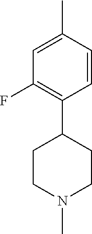 | (M + 1)⁺ 540.1 | CDCl₃: δ 8.83 (s, 1H), 8.46 - 8.37 (m, 1H), 7.80 - 7.66 (m, 2H), 7.50 - 7.47 (m, 2H), 7.38 - 7.34 (m, 1H), 7.29 - 7.27 (m, 1H), 4.29 - 4.23 (m, 2H), 4.06 - 4.01 (m, 2H), 3.63 - 3.58 (m, 2H), 3.06 (t, J = 12.2 Hz, 2H), 2.77 (s, 3H), 2.38 - 2.25 (m, 3H), 2.03 - 1.98 (m, 2H). |
| 11 | 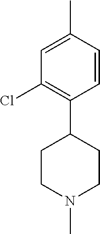 | (M + 1)⁺ 556.2 | CDCl₃: δ 8.84 (s, 1H), 7.96 - 7.92 (m, 1H), 7.50 - 7.47 (m, 2H), 7.43 -7.33 (m, 3H), 7.30 - 7.28 (m, 1H), 4.29 - 4.23 (m, 2H), 4.04 (t, J = 8.8 Hz, 2H), 3.21 - 3.16 (m, 2H), 3.12 - 3.00 (m, 2H), 2.48 (s, 3H), 2.38 - 2.28 (m, 3H), 2.00 - 1.95 (m, 2H). |
| 12 | 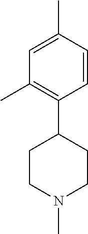 | (M + 1)⁺ 536.3 | CDCl₃: δ 8.82 (s, 1H), 7.66 - 7.45 (m, 4H), 7.40 - 7.33 (m, 2H), 7.29 - 7.27 (m, 1H), 4.28 - 4.19 (m, 2H), 4.03 (t, J = 8.7 Hz, 2H), 3.40 - 3.33 (m, 2H), 2.84 - 2.77 (m, 1H), 2.58 (s, 3H), 2.50 - 2.46 (m, 2H), 2.36 (s, 3H), 2.16 - 2.08 (m, 2H), 1.88 - 1.82 (m, 2H). |
| 13 | 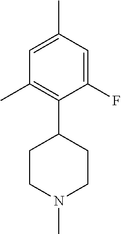 | (M + 1)⁺ 554.3 | CDCl₃: δ 8.82 (s, 1H), 7.69 - 7.51 (m, 2H), 7.49 (d, J = 0.7 Hz, 1H), 7.47 (s, 1H), 7.38 - 7.34 (m, 1H), 6.97 (s, 1H), 4.25 (t, J = 8.7 Hz, 2H), 4.04 (t, J = 8.7 Hz, 2H), 3.22 - 3.16 (m, 2H), 2.84 - 2.78 (m, 1H), 2.48 (s, 3H), 2.45 - 2.39 (m, 2H), 2.37 (s, 3H), 2.33 - 2.25 (m, 2H), 1.74 - 1.68 (m, 2H). |

### Example 14

### Determination of the Inhibitory Effect of Compounds on Wee1 Kinase Using Wee1(h) Kinase Activity Experiment

Wee1 (h) is incubated with 20 mM Tris/HCl pH 8.5, 0.2 mM EDTA, 500 μM LSNLYHQGKFLQTFCGSPLYRRR, 10 mM MgAcetate and 10 μM [γ-³³P]-ATP. Then 50 times the concentration of the testing compound in 100% DMSO was added until the final concentration was 10 μM, then mixed, and diluted to 10 concentrations in successive series according to the ratio of 1:3 and 1:10 respectively (the last concentration was DMSO negative control): 10 μM, 3 μM, 1 μM, 0.3 μM, 0.1 μM, 0.03 μM, 0.01 μM, 0.003 μM, 0.001 μM, 0 μM. The reaction is initiated by the addition of the Mg/ATP mix. After incubation for 40 minutes at room temperature, the reaction is stopped by the addition of phosphoric acid to a concentration of 0.5%. 10 μL of the reaction liquor is then spotted onto a P30 filtermat and washed four times in 0.425% phosphoric acid and once in methanol prior to drying and scintillation counting. Each compound sample is duplicated in duplicate. The negative control was the components except of Wee1 enzyme, and the positive control was the components of adding 30% phosphoric acid to terminate the reaction. Table 1 summarizes the inhibitory effects of compounds on Wee1 kinase activity (IC₅₀).

TABLE 1

| Example | 1 | 2 | 8 | 9 | 12 | 13 | E64* | E70* | E77* |
|---|---|---|---|---|---|---|---|---|---|
| IC₅₀ (nM) | 37 | 21 | 30 | 23 | 23 | 26 | 30 | 48 | 23 |

*Note:
E64, E70 and E77 are compounds of Examples 64, 70 and 77 in WO 2018/090939, respectively.

Therefore, as determined by the Wee1(h) kinase experiment, the compounds of invention (Example 1-13) show good inhibitory effect on Wee1 kinase.

### Example 15

### Determination of the Inhibitory Effect of Compounds on the Proliferation of LoVo Cell Using CCK-8 Method

The thawed LoVo cells were cultured and passaged until they grew well and had a confluence about 90%, and then they were used for experiments. The cells were digested by trypsinase and centrifuged at 800 rpm for 5 minutes, the supernatant was discarded, and the residual was resuspended with fresh medium and counted. The cells were seeded into 96-well cell culture plate with a density of 6000 cells per well and incubated overnight in a 5% C₀₂ incubator at 37° C. The stock solutions of test compounds were serially diluted to 8 concentrations by DMSO at the ratios of 1:3 and 1:10, respectively. The final concentration of compound was: 10 μM, 3.3 μM, 1 μM, 0.33 μM, 0.1 μM, 0.033 μM, 0.01 μM and 0 μM. 5 μL diluent of each concentration was added to 120 μL of medium (25 times diluted) and mixed by shaking. The overnight cell plates were taken and the culture medium was removed, 195 μL of fresh medium was added to each well, and 5 μL of diluted medium containing the corresponding concentration of the test compound was added respectively (the final concentration of DMSO is 1‰), and the culture plate was then placed in a 5% CO₂ incubator at 37° C. for 3 days. After removing the original solution, 90 μL of fresh serum-free 1640 medium was added, then 10 μL of CCK-8 reagent was added to each well and continued incubation for 2 hours. The 96-well plates were placed in a multi-function reader to read the absorbance at the wavelength of 450/650 nm. Cell viability (%)=(OD_{compound}−OD_{background})/(OD_{DMSO}−OD_{background})×100. GraphPad Prism 5.0 was used to analyze the data. The inhibitory activity of compounds on cell proliferation was plotted based on cell viability and the logarithm of compound concentration. The IC₅₀ value was fitted by a sigmoidal dose response curve equation Y=100/(1+10\hat{ }(Log C−Log IC₅₀)), wherein C was the concentration of compound.

Table 2 summarizes the inhibitory effect data (IC₅₀) of compounds on the proliferation of LoVo cell

TABLE 2

| Example | 1 | 2 | 3 | 4 | 5 | 6 | 7 | 8 |
|---|---|---|---|---|---|---|---|---|
| IC₅₀ (μM) | 0.126 | 0.158 | 0.219 | 0.222 | 0.124 | 0.114 | 0.137 | 0.163 |
| Example | 9 | 10 | 11 | 12 | 13 | E47* | E51* | E64* |
| IC₅₀ (μM) | 0.533 | 0.220 | 0.175 | 0.078 | 0.073 | 0.384 | 0.359 | 0.421 |

| Example | E70* | E77* | E78* | E114* | E137* |
|---|---|---|---|---|---|
| IC₅₀ (μM) | 0.557 | 0.166 | 0.204 | 0.662 | 0.757 |

*Note:
E47, E51, E64, E70, E77, E78, E114 and E137 are compounds of Examples 47, 51, 64, 70, 77, 78, 114 and 137 in WO 2018/090939, respectively.

The results in Table 2 showed that Compound 2-8 with one or both of R₃ and R₇ being H had significantly lower IC₅₀ values than compound E64 with both of R₃ and R₇ being H. Compound 2-8 with R₃ or R₇ being either an alkyl or bromine, also had significantly lower IC₅₀ value than Compound E70 with R₃ being F and R₇ being H. Compared with compound E70 with R₃ being methyl and R₇ being H, Compound 5-7 with R₃ and R₇ being alkyl, alkoxy and F, also had significantly lower IC₅₀ values. Compared with Compound E114 with both of R₃ and R₇ being H, Compound 9-13 with one or both of R₃ and R₇ being H had significantly lower IC₅₀ value.

Therefore, as determined by the CCK-8 method, the compounds of invention (Example 1-13) showed inhibitory effect against proliferation of LoVo cell.

### Example 16

### Determination of the Inhibitory Effect of Compounds on the Proliferation of NCI-H1299 Cell Using CCK-8 Method

The thawed NCI-H1299 cells were cultured and passaged until they grew well and had a confluence about 90%, and then they were used for experiments. The cells were digested by trypsinase and centrifuged at 800 rpm for 5 minutes, the supernatant was discarded, and the residual was resuspended with fresh medium and counted. The cells were seeded into 96-well cell culture plate with a density of 1000 cells per well and incubated overnight in a 5% CO₂ incubator at 37° C. The stock solutions of test compounds were serially diluted to 8 concentrations by DMSO at the ratios of 1:3 and 1:10, respectively. The final concentration of compound was: 10 μM, 3.3 μM, 1 μM, 0.33 μM, 0.1 μM, 0.033 μM, 0.01 μM and 0 μM. 5 μL diluent of each concentration was added to 120 μL of medium (25 times diluted) and mixed by shaking. The overnight cell plates were taken and the culture medium was removed, 195 μL of fresh medium was added to each well, and 5 μL of diluted medium containing the corresponding concentration of the test compound was added respectively (the final concentration of DMSO is 1‰), and the culture plate was then placed in a 5% CO₂ incubator at 37° C. for 3 days. After removing the original solution, 90 μL of fresh serum-free 1640 medium was added, then 10 μL of CCK-8 reagent was added to each well and continued incubation for 2 hours. The 96-well plates were placed in a multi-function reader to read the absorbance at the wavelength of 450/650 nm. Cell viability (%)=(OD_{compound}−OD_{background})/(OD_{DMSO}−OD_{background})×100. GraphPad Prism 5.0 was used to analyze the data. The inhibitory activity of compounds on cell proliferation was plotted based on cell viability and the logarithm of compound concentration. The IC₅₀ value was fitted by a sigmoidal dose response curve equation Y=100/(1+10\hat{ }(Log C−Log IC₅₀)), wherein C was the concentration of compound.

Table 3 summarizes the inhibitory effect data (IC₅₀) of compounds on the proliferation of NCI-H1299 cell.

TABLE 3

| Example | 1 | 2 | 3 | 4 | 5 | 6 | 7 | 8 |
|---|---|---|---|---|---|---|---|---|
| IC₅₀ (μM) | 0.071 | 0.123 | 0.382 | 0.838 | 0.182 | 0.140 | 0.209 | 0.214 |
| Example | 9 | 10 | 11 | 12 | 13 | E47* | E51* | E64* |
| IC₅₀ (μM) | 0.940 | 0.204 | 0.166 | 0.079 | 0.085 | 0.574 | 0.396 | 0.315 |

| Example | E70* | E77* | E78* | E114* | E137* |
|---|---|---|---|---|---|
| IC₅₀ (μM) | 0.398 | 0.122 | 0.151 | 0.465 | 0.364 |

*Note:
E47, E51, E64, E70, E77, E78, E114 and E137 are compounds of Examples 47, 51, 64, 70, 77, 78, 114 and 137 in WO 2018/090939, repectively.

Therefore, as determined by the CCK-8 method, the compounds of invention (Example 1-13) show good inhibitory effect against proliferation of NCI-H1299 cell.

Having now fully described this disclosure, it will be understood by those of ordinary skill in the art that the same can be performed within a wide and equivalent range of conditions, formulations and other parameters without affecting the scope of the disclosure or any embodiment thereof. All patents, patent applications and publications cited herein are fully incorporated by reference herein in their entirety.

1. A compound having Formula (I): 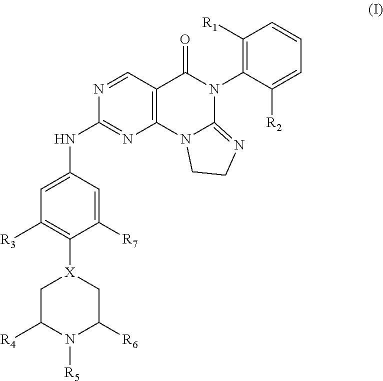
or a stereoisomer thereof, or a pharmaceutically acceptable salt or prodrug thereof, wherein:
R₁ and R₂ are independently halo;
R₃ is halo, C₁₋₄ alkyl or C₁₋₄ alkoxy;
R₄ and R₆ are independently H or C₁₋₄ alkyl;
R₅ is H or C₁₋₄ alkyl;
R₇ is H, halo, C₁₋₄ alkyl or C₁₋₄ alkoxy; and
X is CH or N;
wherein the compound is not:
6-(2-chloro-6-fluorophenyl)-2-((3-fluoro-4-((3R,5S)-3,4,5-trimethylpiperazin-1-yl)phenyl) amino)-8,9-dihydroimidazo[1,2-a]pyrimido[5,4-e]pyrimidin-5(6H)-one;
6-(2-chloro-6-fluorophenyl)-2-((3-chloro-4-((3R,5S)-3,4,5-trimethylpiperazin-1-yl)phenyl)-8,9-dihydroimidazo[1,2-a]pyrimido[5,4-e]pyrimidin-5(6H)-one;
6-(2-chloro-6-fluorophenyl)-2-((3-methyl-4-((3R,5S)-3,4,5-trimethylpiperazin-1-yl)phenyl) amino)-8,9-dihydroimidazo[1,2-a]pyrimido[5,4-e]pyrimidin-5(6H)-one;
6-(2-chloro-6-fluorophenyl)-2-((4-((3S,5R)-4-isopropyl-3,5-dimethylpiperazin-1-yl)-3-methylphenyl)amino)-8,9-dihydroimidazo[1,2-a]pyrimido[5,4-e]pyrimidin-5(6H)-one;
6-(2,6-dichlorophenyl)-2-((3-fluoro-4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-chloro-4-((3S,5R)-3,4,5-trimethylpiperazin-1-yl)phenyl) amino)-8,9-dihydroimidazo[1,2-a]pyrimido[5,4-e]pyrimidin-5(6H)-one;
6-(2,6-dichlorophenyl)-2-((3-methyl-4-((3S,5R)-3,4,5-trimethylpiperazin-1-yl)phenyl) amino)-8,9-dihydroimidazo[1,2-a]pyrimido[5,4-e]pyrimidin-5(6H)-one;
6-(2,6-dichlorophenyl)-2-((3-methyl-4-((3S,5R)-4-isopropyl-3,5-dimethylpiperazin-1-yl) phenyl) amino)-8,9-dihydroimidazo[1,2-a]pyrimido[5,4-e]pyrimidin-5(6H)-one;
6-(2,6-dichlorophenyl)-2-((3,5-dichloro-4-((3S,5R)-3,4,5-trimethylpiperazin-1-yl)phenyl) amino)-8,9-dihydroimidazo[1,2-a]pyrimido[5,4-e]pyrimidin-5(6H)-one; or
6-(2,6-dichlorophenyl)-2-((3-chloro-5-methyl-4-((3S,5R)-3,4,5-trimethylpiperazin-1-yl)phenyl) amino)-8,9-dihydroimidazo[1,2-a]pyrimido[5,4-e]pyrimidin-5(6H)-one.

2. The compound of claim 1 having Formula (Ia): 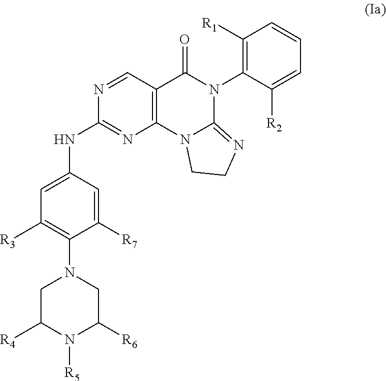
or a stereoisomer thereof, or a pharmaceutically acceptable salt or prodrug thereof, wherein:
R₃ is halo or C₁₋₄ alkyl; and
R₄ and R₆ each are independently C₁₋₄ alkyl.

3. The compound of claim 2, or a stereoisomer thereof or a pharmaceutically acceptable salt or prodrug thereof, wherein:
R₁ and R₂ are chloro;
R₃ is halo, methyl or ethyl;
R₄ and R₆ are methyl;
R₅ is H, methyl or methyl-d3; and
R₇ is H, halo, methyl or methoxy.

4. The compound of claim 1 having Formula (Ib): 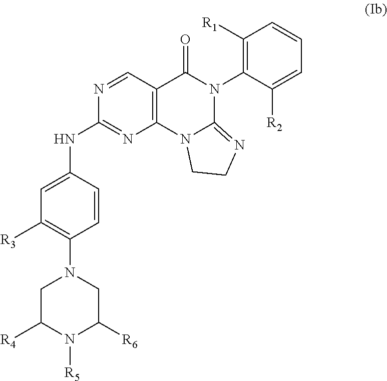
a stereoisomer thereof or a pharmaceutically acceptable salt or prodrug thereof, wherein:
R₃ is C₁₋₄ alkyl;
R₄ and R₆ are independently C₁₋₄ alkyl; and
R₅ is H or C₁₋₄ alkyl, and wherein the alkyl group contains at least 3 deuterium (D).

5. The compound of claim 4, or a stereoisomer thereof, or a pharmaceutically acceptable salt or prodrug thereof, wherein:
R₁ and R₂ are chloro;
R₃ is methyl or ethyl;
R₄ and R₆ are independently methyl; and
R₅ is H or methyl-d3.

6. The compound of claim 1 having Formula (Ic): 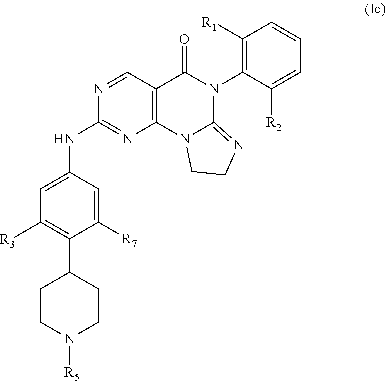
or a stereoisomer thereof, or a pharmaceutically acceptable salt or prodrug thereof.

7. The compound of claim 6, or a stereoisomer thereof, or a pharmaceutically acceptable salt or prodrug thereof, wherein:
R₁ and R₂ are chloro;
R₃ is halo or C₁₋₄ alkyl;
R₅ is C₁₋₄ alkyl; and
R₇ is H or halo.

8. The compound of claim 6, or a stereoisomer thereof, or a pharmaceutically acceptable salt or prodrug thereof, wherein:
R₁ and R₂ are chloro;
R₃ is halo, methyl or ethyl;
R₅ is H, methyl or methyl-d3; and
R₇ is H, halo, methyl or methoxy.

9. The compound of claim 6, or a stereoisomer thereof, or a pharmaceutically acceptable salt or prodrug thereof, wherein:
R₁ and R₂ are chloro;
R₃ is methyl or ethyl;
R₅ is methyl or methyl-d3; and
R₇ is H or halo.

10. The compound of claim 1, or a stereoisomer thereof, or a pharmaceutically acceptable salt or prodrug thereof, wherein the said compound is selected from the group consisting of:

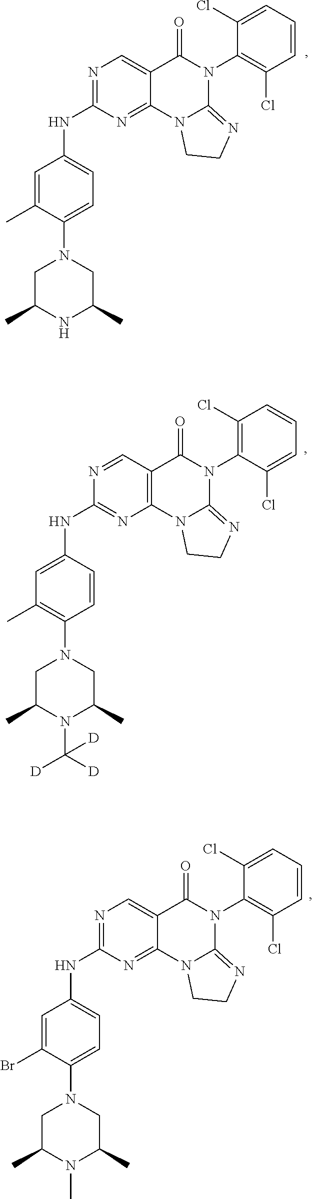 [SMILES: Cc1cc(Nc2ncc3c(n2)N2CCN=C2N(c2c(Cl)cccc2Cl)C3=O)ccc1N1C[C@@H](C)N[C@@H](C)C1 ; [2H]C([2H])([2H])N1[C@H](C)CN(c2ccc(Nc3ncc4c(n3)N3CCN=C3N(c3c(Cl)cccc3Cl)C4=O)cc2C)C[C@@H]1C ; C[C@@H]1CN(c2ccc(Nc3ncc4c(n3)N3CCN=C3N(c3c(Cl)cccc3Cl)C4=O)cc2Br)C[C@H](C)N1C]

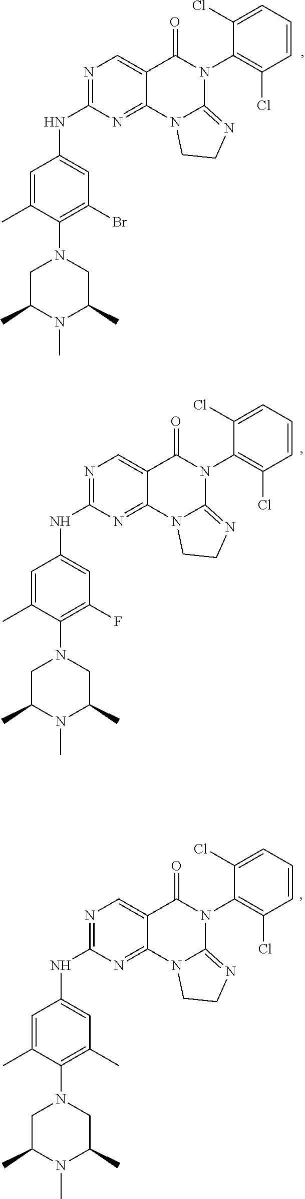 [SMILES: Cc1cc(Nc2ncc3c(n2)N2CCN=C2N(c2c(Cl)cccc2Cl)C3=O)cc(Br)c1N1C[C@@H](C)N(C)[C@@H](C)C1 ; Cc1cc(Nc2ncc3c(n2)N2CCN=C2N(c2c(Cl)cccc2Cl)C3=O)cc(F)c1N1C[C@@H](C)N(C)[C@@H](C)C1 ; Cc1cc(Nc2ncc3c(n2)N2CCN=C2N(c2c(Cl)cccc2Cl)C3=O)cc(C)c1N1C[C@@H](C)N(C)[C@@H](C)C1]

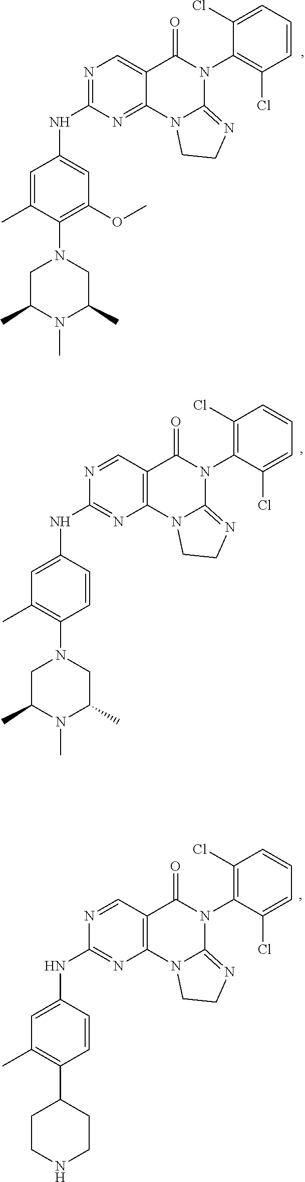 [SMILES: COc1cc(Nc2ncc3c(n2)N2CCN=C2N(c2c(Cl)cccc2Cl)C3=O)cc(C)c1N1C[C@@H](C)N(C)[C@@H](C)C1 ; Cc1cc(Nc2ncc3c(n2)N2CCN=C2N(c2c(Cl)cccc2Cl)C3=O)ccc1N1C[C@H](C)N(C)[C@@H](C)C1 ; Cc1cc(Nc2ncc3c(n2)N2CCN=C2N(c2c(Cl)cccc2Cl)C3=O)ccc1C1CCNCC1]

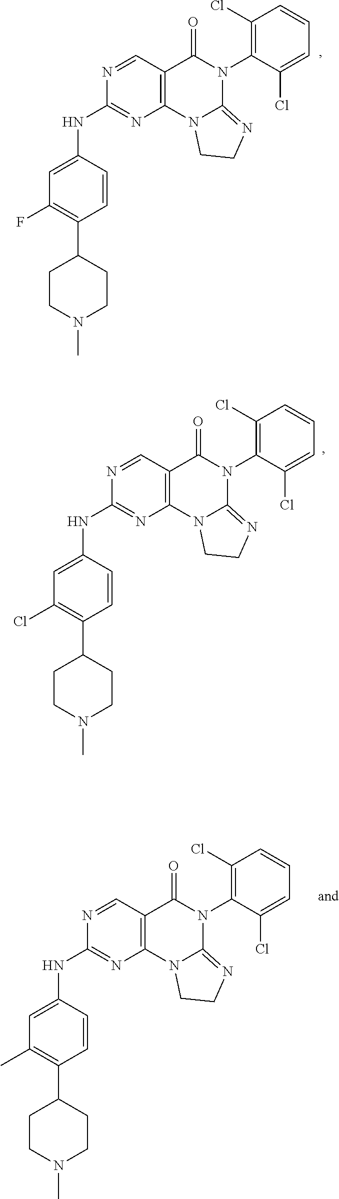 [SMILES: CN1CCC(c2ccc(Nc3ncc4c(n3)N3CCN=C3N(c3c(Cl)cccc3Cl)C4=O)cc2F)CC1 ; CN1CCC(c2ccc(Nc3ncc4c(n3)N3CCN=C3N(c3c(Cl)cccc3Cl)C4=O)cc2Cl)CC1 ; Cc1cc(Nc2ncc3c(n2)N2CCN=C2N(c2c(Cl)cccc2Cl)C3=O)ccc1C1CCN(C)CC1]

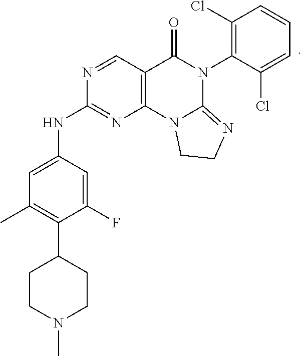 [SMILES: Cc1cc(Nc2ncc3c(n2)N2CCN=C2N(c2c(Cl)cccc2Cl)C3=O)cc(F)c1C1CCN(C)CC1]

11.-13. (canceled)

14. A pharmaceutical composition comprising the compound of any claim 1 and a pharmaceutically acceptable carrier.

15. The pharmaceutical composition of claim 14, further comprising at least one known anticancer agent, or a pharmaceutically acceptable salt of said anticancer agent.

16. The pharmaceutical composition of claim 15, wherein the at least one anticancer agent is busulfan, melphalan, chlorambucil, cyclophosphamide, ifosfamide, temozolomide, bendamustine, cis-platin, mitomycin C, bleomycin, carboplatin, camptothecin, irinotecan, topotecan, doxorubicin, epirubicin, aclarubicin, mitoxantrone, elliptinium, etoposide, 5-azacytidine, gemcitabine, 5-fluorouracil, methotrexate, 5-fluoro-2′-deoxy-uridine, fludarabine, nelarabine, ara-C, pralatrexate, pemetrexed, hydroxyurea, thioguanine, colchicine, vinblastine, vincristine, vinorelbine, paclitaxel, ixabepilone, cabazitaxel, docetaxel, campath, panitumumab, metazotuzumab, navuzumab, pymzumab, remoluzumab, bevacizumab, partuzumab, trastuzumab, cetuximab, obinutuzumab, olfamzumab, rituximab, alemtuzumab, tiemuzumab, toximab, bentuximab, daremuzumab, errotuzumab, T-DM1, ofatumumab, dinutuximab, blinatumomab, ipilimma, avastin, trastuzumab, rituximab, imatinib, gefitinib, erlotinib, osimertinib, afatinib, ceritinib, aletinib, crizotinib, erlotinib, lapatinib, sorafenib, sunitinib, nilotinib, dasatinib, pazopanib, temsirolimus, everolimus, vorinostat, romidepsin, panobinostat, belinostat, tamoxifen, letrozole, fulvestrant, mitoguazone, octreotide, retinoic acid, arsenic trioxide, zoledronic acid, bortezomib, carfilzomib, ixazomib, vismodegib, sonidegib, denosumab, thalidomide, lenalidomide, venetoclax, aldesleukin (recombinant human interleukin-2), sipueucel-T (prostate cancer therapeutic vaccine), palbociclib, olaparib, niraparib, rucaparib, talazoparib or senaparib.

17. The pharmaceutical composition of claim 14, wherein the compound is a compound having Formula (Ia): 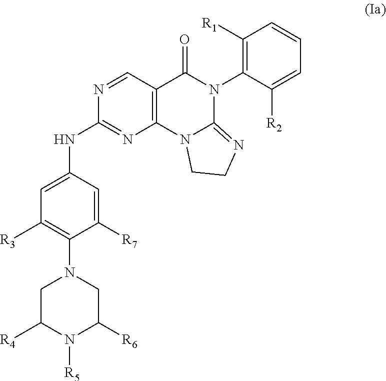
or a stereoisomer thereof, or a pharmaceutically acceptable salt or prodrug thereof, wherein:
R₃ is halo or C₁₋₄ alkyl; and
R₄ and R₆ are independently C₁₋₄ alkyl; or
the compound is a compound having Formula (Ib): 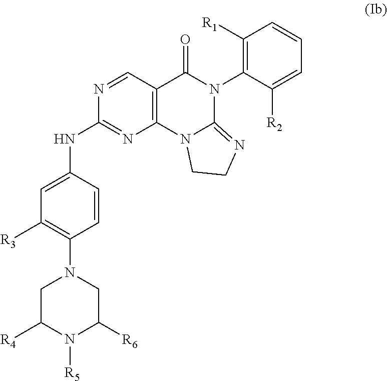
or a stereoisomer thereof, or a pharmaceutically acceptable salt or prodrug thereof, wherein:
R₃ is C₁₋₄ alkyl;
R₄ and R₆ are independently C₁₋₄ alkyl; and
R₅ is H or C₁₋₄ alkyl, wherein the alkyl group contains at least 3 deuterium (D); or
the compound is a compound having Formula (Ic): 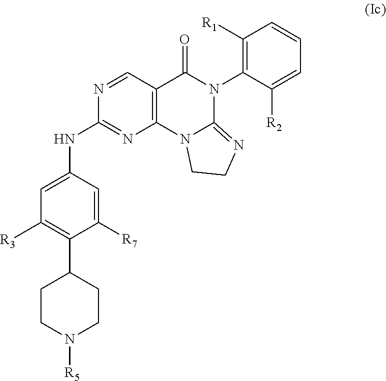
or a stereoisomer thereof, or a pharmaceutically acceptable salt or prodrug thereof.

18. The pharmaceutical composition of claim 14, wherein the compound is selected from the group consisting of:

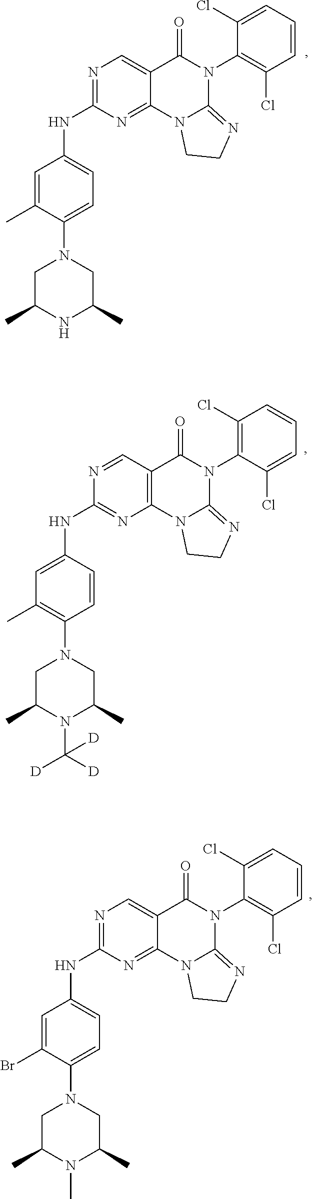 [SMILES: Cc1cc(Nc2ncc3c(n2)N2CCN=C2N(c2c(Cl)cccc2Cl)C3=O)ccc1N1C[C@@H](C)N[C@@H](C)C1 ; [2H]C([2H])([2H])N1[C@H](C)CN(c2ccc(Nc3ncc4c(n3)N3CCN=C3N(c3c(Cl)cccc3Cl)C4=O)cc2C)C[C@@H]1C ; C[C@@H]1CN(c2ccc(Nc3ncc4c(n3)N3CCN=C3N(c3c(Cl)cccc3Cl)C4=O)cc2Br)C[C@H](C)N1C]

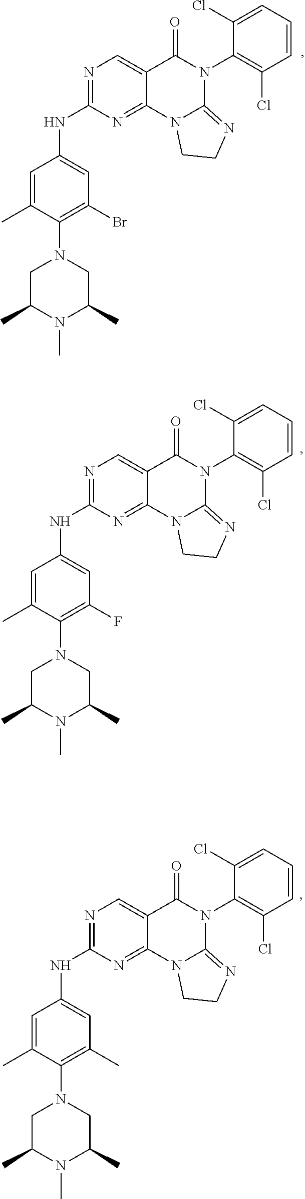 [SMILES: Cc1cc(Nc2ncc3c(n2)N2CCN=C2N(c2c(Cl)cccc2Cl)C3=O)cc(Br)c1N1C[C@@H](C)N(C)[C@@H](C)C1 ; Cc1cc(Nc2ncc3c(n2)N2CCN=C2N(c2c(Cl)cccc2Cl)C3=O)cc(F)c1N1C[C@@H](C)N(C)[C@@H](C)C1 ; Cc1cc(Nc2ncc3c(n2)N2CCN=C2N(c2c(Cl)cccc2Cl)C3=O)cc(C)c1N1C[C@@H](C)N(C)[C@@H](C)C1]

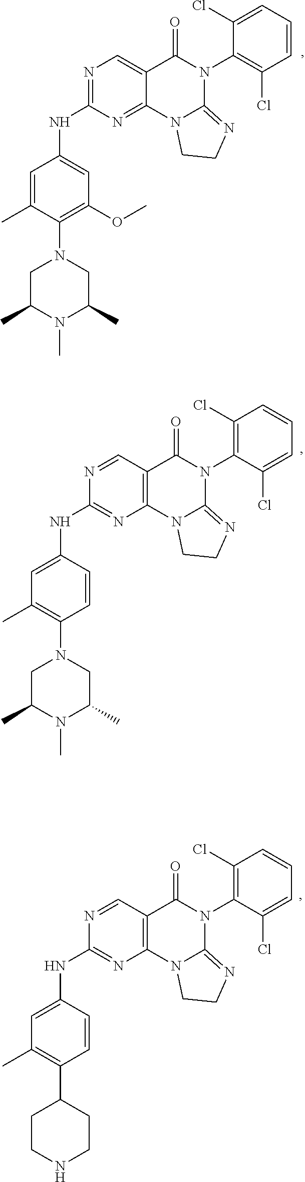 [SMILES: COc1cc(Nc2ncc3c(n2)N2CCN=C2N(c2c(Cl)cccc2Cl)C3=O)cc(C)c1N1C[C@@H](C)N(C)[C@@H](C)C1 ; Cc1cc(Nc2ncc3c(n2)N2CCN=C2N(c2c(Cl)cccc2Cl)C3=O)ccc1N1C[C@H](C)N(C)[C@@H](C)C1 ; Cc1cc(Nc2ncc3c(n2)N2CCN=C2N(c2c(Cl)cccc2Cl)C3=O)ccc1C1CCNCC1]

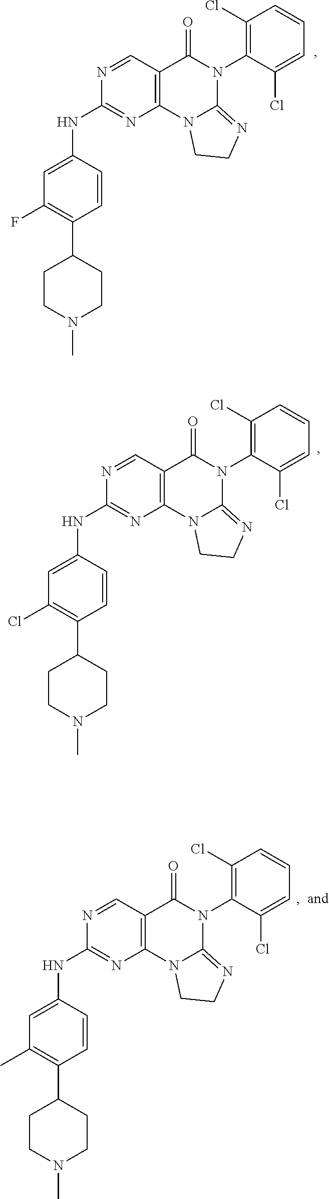 [SMILES: CN1CCC(c2ccc(Nc3ncc4c(n3)N3CCN=C3N(c3c(Cl)cccc3Cl)C4=O)cc2F)CC1 ; CN1CCC(c2ccc(Nc3ncc4c(n3)N3CCN=C3N(c3c(Cl)cccc3Cl)C4=O)cc2Cl)CC1 ; Cc1cc(Nc2ncc3c(n2)N2CCN=C2N(c2c(Cl)cccc2Cl)C3=O)ccc1C1CCN(C)CC1]

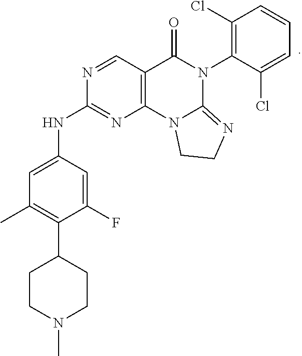 [SMILES: Cc1cc(Nc2ncc3c(n2)N2CCN=C2N(c2c(Cl)cccc2Cl)C3=O)cc(F)c1C1CCN(C)CC1]

19. A method for treating or preventing a Wee1-mediated disease in a subject in need thereof, comprising administering to the subject an effective amount of a compound of claim 1, or a stereoisomer thereof, or a pharmaceutically acceptable salt or prodrug thereof, or a pharmaceutical composition comprising the compound, or a stereoisomer thereof, or a pharmaceutically acceptable salt or prodrug thereof.

20. The method of claim 19, wherein the disease is cancer.

21. The method of claim 20, wherein the cancer is liver cancer, melanoma, Hodgkin's disease, non-Hodgkin's lymphomas, acute lymphocytic leukemia, chronic lymphocytic leukemia, multiple myeloma, neuroblastoma, breast carcinoma, ovarian carcinoma, lung carcinoma, Wilms' tumor, cervical carcinoma, testicular carcinoma, soft-tissue sarcoma, chronic lymphocytic leukemia, primary macroglobulinemia, bladder carcinoma, chronic granulocytic leukemia, primary brain carcinoma, malignant melanoma, small-cell lung carcinoma, stomach carcinoma, colon carcinoma, malignant pancreatic insulinoma, malignant carcinoid carcinoma, malignant melanoma, choriocarcinoma, mycosis fungoide, head and neck carcinoma, osteogenic sarcoma, pancreatic carcinoma, acute granulocytic leukemia, hairy cell leukemia, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, adrenal cortex carcinoma, skin cancer, or prostatic carcinoma.

22. The method of claim 19, wherein the compound is a compound having Formula (Ia): 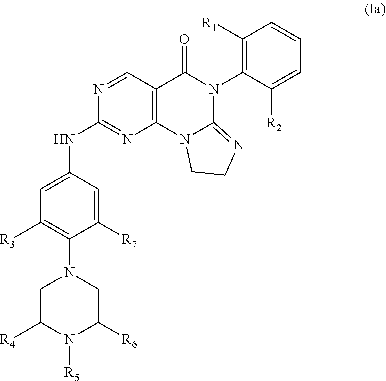
or a stereoisomer thereof, or a pharmaceutically acceptable salt or prodrug thereof, wherein:
R₃ is halo or C₁₋₄ alkyl; and
R₄ and R₆ are independently C₁₋₄ alkyl; or
the compound is a compound having Formula (Ib): 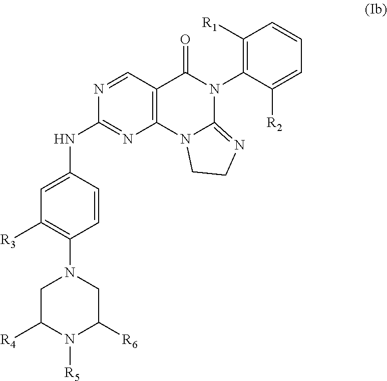
or a stereoisomer thereof, or a pharmaceutically acceptable salt or prodrug thereof, wherein:
R₃ is C₁₋₄ alkyl;
R₄ and R₆ are independently C₁₋₄ alkyl; and
R₅ is H or C₁₋₄ alkyl, wherein the alkyl group contains at least 3 deuterium (D); or
the compound is a compound having Formula (Ic): 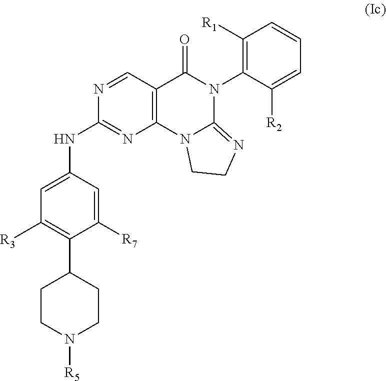
or a stereoisomer thereof, or a pharmaceutically acceptable salt or prodrug thereof.

23. The method of claim 19, wherein the compound is selected from the group consisting of: 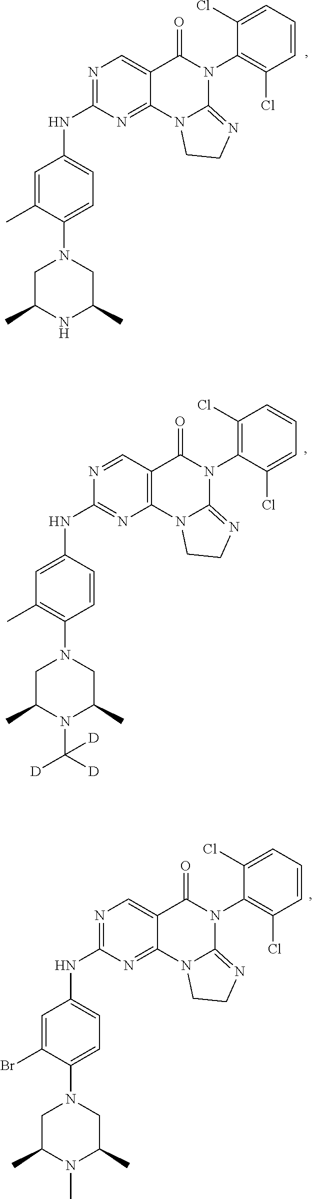 [SMILES: Cc1cc(Nc2ncc3c(n2)N2CCN=C2N(c2c(Cl)cccc2Cl)C3=O)ccc1N1C[C@@H](C)N[C@@H](C)C1 ; [2H]C([2H])([2H])N1[C@H](C)CN(c2ccc(Nc3ncc4c(n3)N3CCN=C3N(c3c(Cl)cccc3Cl)C4=O)cc2C)C[C@@H]1C ; C[C@@H]1CN(c2ccc(Nc3ncc4c(n3)N3CCN=C3N(c3c(Cl)cccc3Cl)C4=O)cc2Br)C[C@H](C)N1C] 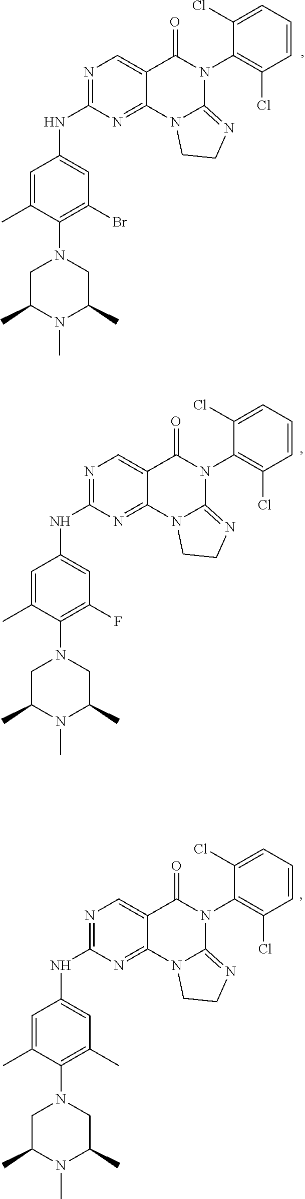 [SMILES: Cc1cc(Nc2ncc3c(n2)N2CCN=C2N(c2c(Cl)cccc2Cl)C3=O)cc(Br)c1N1C[C@@H](C)N(C)[C@@H](C)C1 ; Cc1cc(Nc2ncc3c(n2)N2CCN=C2N(c2c(Cl)cccc2Cl)C3=O)cc(F)c1N1C[C@@H](C)N(C)[C@@H](C)C1 ; Cc1cc(Nc2ncc3c(n2)N2CCN=C2N(c2c(Cl)cccc2Cl)C3=O)cc(C)c1N1C[C@@H](C)N(C)[C@@H](C)C1] 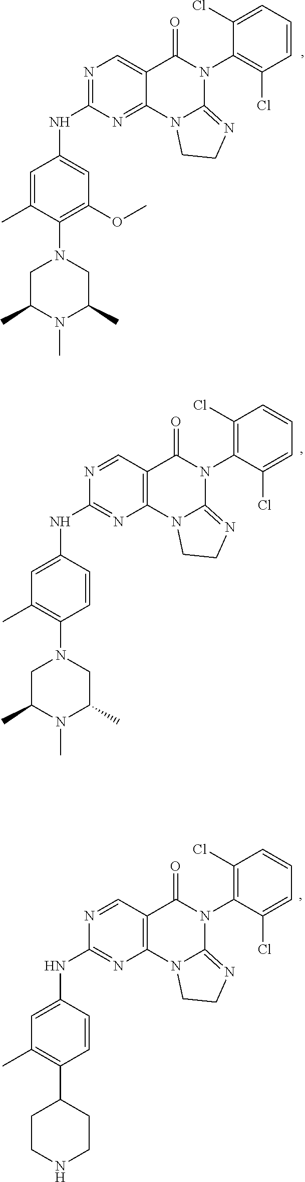 [SMILES: COc1cc(Nc2ncc3c(n2)N2CCN=C2N(c2c(Cl)cccc2Cl)C3=O)cc(C)c1N1C[C@@H](C)N(C)[C@@H](C)C1 ; Cc1cc(Nc2ncc3c(n2)N2CCN=C2N(c2c(Cl)cccc2Cl)C3=O)ccc1N1C[C@H](C)N(C)[C@@H](C)C1 ; Cc1cc(Nc2ncc3c(n2)N2CCN=C2N(c2c(Cl)cccc2Cl)C3=O)ccc1C1CCNCC1] 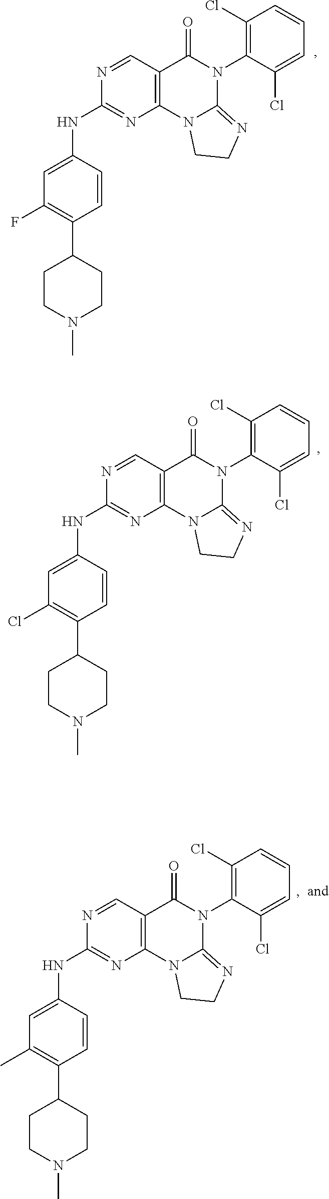 [SMILES: CN1CCC(c2ccc(Nc3ncc4c(n3)N3CCN=C3N(c3c(Cl)cccc3Cl)C4=O)cc2F)CC1 ; CN1CCC(c2ccc(Nc3ncc4c(n3)N3CCN=C3N(c3c(Cl)cccc3Cl)C4=O)cc2Cl)CC1 ; Cc1cc(Nc2ncc3c(n2)N2CCN=C2N(c2c(Cl)cccc2Cl)C3=O)ccc1C1CCN(C)CC1] 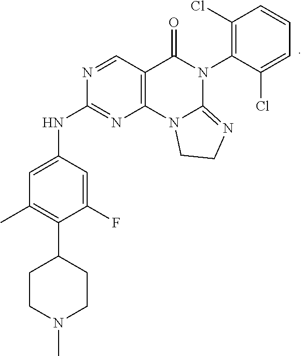 [SMILES: Cc1cc(Nc2ncc3c(n2)N2CCN=C2N(c2c(Cl)cccc2Cl)C3=O)cc(F)c1C1CCN(C)CC1]
